(19) United States
(12) Patent Application Publication
(10) Pub. No.: US 2023/0389784 A1
(43) Pub. Date: Dec. 7, 2023

(54) SYSTEMS AND METHODS FOR ENDOSCOPIC SURGERY

(71) Applicant: Theragi LLC, Trumbull, CT (US)

(72) Inventor: Oleg Shikhman, et al., Trumbull, CT (US)

(73) Assignee: Theragi LLC, Trumbull, CT (US)

(21) Appl. No.: 18/029,145

(22) PCT Filed: Oct. 6, 2021
(86) PCT No.: PCT/US2021/053685

§ 371 Date: Mar. 29, 2023

Related U.S. Application Data
(60) Provisional application No. 63/091,121, filed on Oct. 13, 2020.
(60) Provisional application No. 63/118,950, filed on Nov. 29, 2020.
(60) Provisional application No. 63/138,949, filed on Jan. 19, 2021.
(60) Provisional application No. 63/170,595, filed on Apr. 5, 2021.

(51) Int. Cl. A61B 1/018; A61B 1/00; A61B 17/10; A61B 1/005

(52) U.S. Cl. CPC A61B 1/018; A61B 1/00128; A61B 17/10; A61B 1/005; A61B 1/00137

(57) ABSTRACT

A system for tissue resection during endoscopic surgery having an endoscope connector connectable to an endoscope and an instrument receiving channel dimensioned and configured to receive an endoscopic instrument. The endoscope connector connects the instrument receiving channel external of the endoscope. A second channel can be provided radially spaced from the first channel for insertion of a second endoscopic instrument. Devices for tissue tensioning are also disclosed.

### BACKGROUND OF THE INVENTION

This application claims priority to provisional application 63/091,121, filed Oct. 13, 2020, provisional application 63/118,950, filed Nov. 29, 2020, provisional application 63/138,949, filed Jan. 19, 2021, and provisional application 63/170,595, filed Apr. 5, 2021. The entire contents of each of these applications are incorporated herein by reference.

### 1. FIELD OF THE INVENTION

This application relates to systems for endoscopic resections, and, more particularly, to systems mountable to endoscopes containing more or more channels for instrumentation to access target tissue.

### 2. BACKGROUND

When performing a resection in traditional open or laparoscopic surgery, a surgeon typically manipulates tissue with two instruments. One instrument is a cutting/dissecting device, such as a scalpel, scissors, or an electrosurgical cutting device. The second instrument is a retractor that allows the surgeon to tension tissue that is being cut/dissected. Tissue is easier to cut, when it is under tension. Also, such conventional surgical technique makes cutting safer by pulling tissue away from the cutting point allowing the surgeon to visualize the area of resection and a cutting plane.

Recently, minimally invasive techniques such as endoscopic mucosal resection (EMR), endoscopic submucosal dissection (ESD) and endoscopic full-thickness resection (EFTR) are gaining popularity. During EMR, a physician dissects mucosal tissue superficially, while during ESD, the dissection penetrates deeper layers of the wall, such as submucosa. EFTR procedure allows the physician to go through the wall completely. These procedures are less invasive than traditional open or laparoscopic surgeries because they don't require an opening in the abdominal wall. Instead, they are performed via the body lumens, for example, a colon with a trans-anal approach or an esophagus trans-orally. Regardless, these procedures are surgical in nature, and a stable and reliable tissue traction is still necessary.

In these procedures, flexible endoscopic instruments are inserted through working channels of the endoscope. The instruments are articulated by articulation of the endoscope. Since the instrument articulation is controlled by movement of the endoscope, maneuverability of the instruments is limited and/or constrained/defined by the scope maneuvers. Additionally, since the instruments are advanced distally of the scope, they are approaching the lesion at a more direct angle with limited space.

The need exists to improve the maneuverability and accessibility of flexible endoscopic instruments within confined body working spaces such as the colon or other body lumens.

It would be beneficial if such systems enable movement of the endoscopic instruments independent of endoscope movement.

Additionally, in current systems, several hands are required to perform the various functions of the surgical procedure. These functions include advancing the endoscope, articulating the endoscope, advancing various endoscopic instruments through the working channels of the endoscope and actuating the endoscopic instruments, e.g., closing jaws of the grasper on tissue, maneuvering tissue dissection instruments, etc. Thus, these procedures require more than the two hands of the clinician and require the additional hands of an assistant.

It would be advantageous to provide a system to effectively enable maneuverability and actuation of endoscopic instruments with a single hand of the clinician during endoscopic resection procedures. Such systems would simplify the procedure. Such systems would also benefit other surgical procedures. It would also be beneficial to improve tissue traction during minimally invasive procedures which would facilitate dissection of tissue.

It would be beneficial to provide such systems for endoscopic procedures in the upper gastrointestinal tract, for example, the esophagus, stomach, duodenum, and the lower gastrointestinal tract, for example, rectum, sigmoid, colon, appendix.

The need therefore exists for a system for performing endoscopic resections in the GI tract, as well as for other clinical applications, that effectively organize the various instrumentation, improve access to the target tissue, improve visibility of the target tissue, improve maneuverability of the instrumentation, improve tissue traction to aid dissection, and can be easily controlled by the clinician.

### SUMMARY

The present invention overcomes the problems and deficiencies of the prior art. The present invention advantageously provides devices, systems and methods for facilitating endoscopic resections in the GI tract or other regions of the patient's body.

The systems of the present invention include an endoscope connector and an instrument receiving channel. In some embodiments, the endoscope connector is in the form of hub, cap, or sheath mountable over the endoscope. In other embodiments, the endoscope connector is in the form of a strap, tape, etc. wrapped around the endoscope. Other connectors are also contemplated to achieve the same functions. In these various embodiments, the endoscope connector of the systems of the present invention connects (attaches/mounts) the instrument receiving channel to the endoscope in a position external of the endoscope such that the instrument receiving channel remains external of the endoscope during insertion and use.

The instrument receiving channel forms an instrument guide for the endoscopic instrument either directly or indirectly. That is, the instrument receiving channel can directly receive the endoscopic instrument or the instrument receiving channel can receive an additional instrument guide and the additional instrument guide receives the endoscopic instrument. This is also described below. Thus, the use herein of the term “guide” or “instrument guide” refers to either a) the instrument receiving channel (if used without an additional instrument guide) or b) the additional guide if positioned within the instrument receiving channel.

The guide is configured to direct an endoscopic instrument toward the target site at an increased angle as opposed to a more direct angle if the instrument is inserted through a working channel of the endoscope. In some embodiments, the instrument guides are bent via actuation of an actuator by a clinician. In other embodiments, the instrument guides are pre-bent to a desired angle/configuration and automatically move to the pre-bent position when advanced from the confines of the endoscope connector (e.g., hub/cap/sheath) or endoscope connector channel or conduit.

The systems and methods of the present invention in some embodiments provide for movement, e.g., bending, of the instrument guide independent of the articulation (bending) of the endoscope. In some of these embodiments, the user can selectively decide whether to have the instrument guide articulate along with the scope, i.e., tie together the movements of the endoscope and instrument guide, or separate the movements of the endoscope and instrument guide so that articulation of the endoscope does not affect articulation of the instrument guide and bending of the instrument guide can be performed independent of the endoscope position. Thus, the user decides the linking or unlinking conditions of the system.

The endoscope connector in some embodiments includes a hub/cap/sheath which includes an instrument guide that is movable independent of the hub/cap/sheath. The instrument guide can be integral with the hub or a separate component attached thereto. This independent movement can include one or more of a) slidable (axial) movement relative to the hub/cap/sheath; b) bendable (articulation) movement relative to the hub/cap/sheath; c) lateral movement relative to the hub/cap/sheath to increase the distance to the target tissue; and/or c) rotational movement relative to the hub/cap/sheath. Such movements are also relative to the hub/cap/sheath opening for the endoscope. Such relative movements are also applicable to other embodiments of the endoscope connector. In such embodiments, one or more of these movements can be effected independent of the position of the endoscope relative to the hub/cap/sheath.

The endoscope connector of the present invention can in some embodiments also include a second channel, radially spaced from the instrument guide channel for passage of an endoscopic instrument such as an instrument to stabilize the tissue, e.g., lesion being dissected.

The systems of the present invention can be retrofitted to a conventional endoscope if desired or provided with an endoscope.

In accordance with one aspect of the present invention, a system for tissue resection during endoscopic surgery is provided. The system comprises an instrument receiving channel dimensioned and configured to receive an endoscopic instrument and an endoscope connector connecting the instrument receiving channel to the endoscope external of the endoscope. In a first condition selected by a user, articulation of the endoscope to an angle to a longitudinal axis of the endoscope effects deformation of a distal portion of the instrument receiving channel and in a second condition selected by the user, articulation of the endoscope does not deform the distal portion of the channel such that the channel is deformable independent of the endoscope.

In such systems, the first and second conditions can be different conditions of the instrument receiving channel.

In some embodiments, the connector is in the form of a hub mountable to a distal portion of the endoscope, the hub having an opening configured and dimensioned to receive at least a distal portion of the endoscope.

In some embodiments of such systems, the first and second conditions are achievable independent of the position of the endoscope. In some embodiments of such systems, the instrument receiving channel is bendable, slid able, and/or rotatable relative to the opening in the hub.

In some embodiments, the system includes an endoscope locking member (element) selectively movable/controllable between a locking (engaging) position and a non-locking (disengaging) position to effect the first and second condition, wherein in the locking position the first condition is effected as articulation of the endoscope to an angle to a longitudinal axis of the endoscope bends a distal portion of the channel and in the non-locking position, the second condition is effected as articulation of the endoscope does not bend the distal portion of the channel such that the channel is bendable independent of the endoscope.

The locking member in some embodiments comprises a compression element pivotable between a first position to connect the channel to the endoscope and a second position to disconnect the channel from the endoscope. The compression element can be biased to a locking position.

In some embodiments, the channel in a bent position curves away from the opening in the endoscope connector and then back toward an opening in the endoscope connector.

In some embodiments, the channel is pre-bent and is retained in a more linear position when retracted within a conduit of the endoscope connector and moves to the pre-bent position when exposed from the conduit.

In some embodiments, the channel is actively bent by action of the user. Such embodiments can include a hub including a link connected to the channel and an elongated member operatively connected to the link, wherein actuation of the elongated member effects pivoting of the link to effect bending of the channel. In some embodiments, the hub includes a second channel and an actuator, the elongated member positioned within the second channel and the second channel is radially spaced from the instrument receiving channel, wherein the actuator moves the elongated member within the second channel to tension the elongated member. In some embodiments, the actuator is slidable to tension the elongated member to pivot the link; in other embodiments, the actuator is pivotable to tension the elongated member to pivot the link.

In some embodiments, the hub includes a distal conduit and the instrument receiving channel is slidable within the distal conduit.

In some embodiments, the instrument receiving channel is part of the endoscope connector and receives a flexible instrument guide, the flexible instrument guide movable within the channel from a retracted position to an advanced position. The instrument guide guides the endoscopic instrument extending therethrough. In other embodiments, the instrument receiving channel itself forms the flexible instrument guide to receive the endoscopic instrument.

In some embodiments, the channel has a first opening spaced distally from a distalmost end of the channel, and the flexible guide is advanceable through the first opening. The channel can optionally have a second opening at the distalmost end, and the flexible guide can be selectively insertable through either the first opening or the second opening of the channel wherein insertion through the second opening effects the first condition of tying together the flexible guide and endoscope and insertion through the first opening effects the second condition of independent movement of the flexible guide from the endoscope.

In some embodiments, the hub includes a second channel radially spaced from the instrument receiving channel, the second channel configured and dimensioned to receive a second endoscopic instrument. The system can include the second endoscopic instrument and an actuator operatively connected to the second instrument for advancing and retracting the endoscopic instrument. The second endoscopic instrument can be an instrument for stabilizing tissue or other endoscopic instruments performing other functions.

The system in some embodiments can have a radially extending inflatable balloon to stabilize the system.

In some embodiments, to aid organization of the instrumentation used in the surgical procedure, the endoscope connector includes at least one spacer positioned proximally of a distalmost end, the at least one spacer including a lumen to receive the instrument receiving channel. The at least one spacer can include a second lumen to receive a tissue stabilizing instrument connected to the endoscope connector.

In some embodiments, the system further comprises a clip applying instrument movable within the second channel, the clip applying instrument comprising a pair of jaws for opening and releasing a clip carried between the pair of jaws. The clip in some embodiments can be biased to a closed position and the jaws open the clip for application to tissue and release the clip to return to its normally closed position.

In some embodiments, the system includes a flexible member linking the instrument receiving channel and a portion of the endoscope connector, wherein the flexible member is tensionable to interlock the instrument receiving channel and the portion of the endoscope connector containing the instrument receiving channel so articulation of the endoscope bends the instrument receiving channel, and when the flexible member is not tensioned, articulation of the endoscope does not bend the instrument receiving channel.

In some embodiments, at least a distal portion of the instrument receiving channel is collapsible, wherein insertion of an endoscopic instrument (or of the instrument guide in other embodiments) through the channel expands the channel and in the absence of the endoscopic instrument (or instrument guide), the channel is in a collapsed condition.

In accordance with another aspect of the present invention, a system for tissue resection during endoscopic surgery is provided. The system comprises an endoscope connector configured to connect to an endoscope and an instrument receiving channel. The endoscope connector connects the instrument receiving channel to the endoscope such that the instrument receiving channel is external of the endoscope. The instrument receiving channel is movable with respect to the endoscope connector and is dimensioned and configured to receive an endoscopic instrument. The instrument receiving channel is bendable with respect to a longitudinal axis of the endoscope such that a distal portion of the endoscopic instrument extending distally of the instrument receiving channel bends at an acute angle toward target tissue.

In some embodiments the instrument receiving channel is movable with respect to the endoscope connector or an opening for the endoscope in the endoscope connector, and such movement can include one of more of a) bending; b) sliding (axially and or/laterally); and/or c) rotation.

In some embodiments, the channel is pre-bent to the acute angle. In some embodiments, the system further comprises a link connected to the channel and an elongated member operatively connected to the link, wherein actuation of the elongated member effects pivoting of the link to effect bending of the channel.

In some embodiments, the endoscope connector includes a second channel and an actuator, and the elongated member is positioned within the second channel and the second channel is radially spaced from the instrument receiving channel, wherein the actuator is operatively connected to the elongated member and moves the elongated member within the second channel to tension the elongated member.

In some embodiments, the actuator is slidable to tension the elongated member to pivot the link; in other embodiments, the actuator is pivotable to tension the elongated member to pivot the link.

In some embodiments, the endoscope connector includes a distal conduit and the instrument receiving channel is slid able within the distal conduit.

In some embodiments, the channel in a bent position curves away from the opening and then back toward the opening.

In some embodiments, the instrument receiving channel is part of the endoscope connector and is configured and dimensioned to receive a flexible instrument guide, the flexible instrument guide movable within the channel from a retracted position to an advanced position exposed from the channel.

In some embodiments, the instrument receiving channel has a first opening spaced proximally from a distalmost end of the channel, and the flexible guide is advanceable through the first opening. In some embodiments, the instrument receiving channel has a second opening at or adjacent the distalmost end in addition to the first opening, and the flexible guide is selectively insertable through either the first opening or the second opening of the instrument receiving channel wherein insertion through the second opening ties together articulation of the endoscope and bending of the channel and insertion through the first opening enables bending of the channel (and flexible guide) independent of articulation of the endoscope.

The endoscope connector can include an additional channel radially spaced from the instrument receiving channel, and external of the endoscope, the additional channel configured and dimensioned to receive a second endoscopic instrument such as a tissue stabilizer or tissue retraction device.

In some embodiments, the system includes the second endoscopic instrument and an actuator operatively connected to the second instrument for advancing and retracting the endoscopic instrument, the second endoscopic instrument stabilizing tissue. Other instruments can also be utilized.

The endoscope connector in some embodiments includes at least one spacer positioned proximally of the distalmost end of the hub, the at least one spacer including a lumen to receive the instrument receiving channel. In some embodiments, the at least one spacer includes a second lumen to receive a tissue stabilizing instrument connected to the endoscope connector. Multiple spacers can be provided.

The system can include a clip applying instrument movable within the second channel, the clip applying instrument comprising a pair of jaws for opening and releasing a clip carried between the pair of jaws. The clip can be placed at an angle between about 45 degrees and about 90 degrees to the longitudinal axis of the endoscope. Other angles are also contemplated.

In some embodiments, at least a distal portion of the instrument receiving channel is collapsible, wherein insertion of an endoscopic instrument or instrument guide through the channel expands the channel and in the absence of the endoscopic instrument or instrument guide, the channel is in a collapsed condition.

In accordance with another aspect of the present invention, a system for tissue resection during endoscopic surgery is provided comprising:
a) an endoscope connector mountable (connectable) to an endoscope;
b) an instrument receiving channel, the endoscope connector connecting the instrument receiving channel external of the endoscope;
c) a flexible instrument guide movable with the instrument receiving channel, the flexible instrument channel dimensioned and configured to receive an endoscopic instrument, the instrument receiving channel bendable with respect to a longitudinal axis of the endoscope such that a distal portion of the endoscopic instrument extending distally of the instrument receiving channel bends inwardly at an acute angle toward target tissue; and
d) an endoscopic instrument movable within the flexible instrument guide, the endoscopic instrument advanceable from the instrument guide to angle toward target tissue, wherein the flexible instrument guide and the endoscopic instrument are movable by a single hand of a user.

In some embodiments, the endoscope is articulated to an angle to a longitudinal axis of the endoscope by the other hand of the user.

In some embodiments, the system includes an actuator for moving the flexible instrument guide between retracted and advanced positions. In some embodiments, the guide is retained in position when the user ceases advancement of the guide and/or the endoscopic instrument is retained in position when the user ceases advancement of the endoscopic instrument.

In accordance with another aspect of the present invention, a system for tissue resection during endoscopic surgery is provided, the system comprising:
a) an instrument receiving channel;
b) an endoscope connector connecting the instrument receiving channel external of the endoscope; and
e) an actuator for moving the flexible instrument guide between first and second positions;
f) wherein the instrument receiving channel is retained in position when the user ceases movement if the instrument receiving channel;

In some embodiments, the system further includes a flexible instrument guide movable with the channel, the instrument guide dimensioned and configured to receive an endoscopic instrument, and the instrument guide is bendable with respect to a longitudinal axis of the endoscope such that a distal portion of the endoscopic instrument extending distally of the instrument guide channel bends inwardly at an acute angle toward target tissue. In some embodiments, the instrument guide is retained in an axial and rotational position when the user ceases movement of the instrument guide.

In some embodiments, the system further comprises an endoscopic instrument movable within the instrument guide, the endoscopic instrument advanceable from the instrument guide to angle toward target tissue. In some embodiments, the endoscopic instrument is retained in an axial and rotational position when the user ceases movement of the endoscopic instrument.

The movement of the instrument receiving channel, instrument guide and/or endoscopic instrument can include bending (deforming), axial movement, lateral movement and/or rotational movement.

In some embodiments, the endoscopic instrument is retained in an axial and rotational position when the user ceases advancement of the endoscopic instrument.

In some embodiments, the instrument guide is retained by a frictional engagement of an actuator for the guide.

A control handle for moving the guide axially and rotationally can be provided in some embodiments wherein the control handle is mountable to the endoscope. In other embodiments, the control handle is mounted to a bracket of an operating table.

The present invention also includes methods for utilizing the systems described herein. For example, one method of the present invention provides a method of endoscopically resecting tissue comprising the steps of:
a) mounting an endoscope connector to an endoscope, such that first and second instrument receiving channels are external of the endoscope;
b) advancing a flexible instrument guide so a distal portion extends distally from the first channel, the flexible instrument guide dimensioned and configured to receive an endoscopic tissue grasping instrument, the instrument guide bendable with respect to a longitudinal axis of the endoscope and bendable toward the longitudinal axis of the endoscope;
c) an endoscopic tissue grasping instrument movable within the instrument guide, the endoscopic tissue grasping instrument advanceable from the instrument guide to angle toward target tissue;
d) advancing a clip applying instrument carrying a clip through the second channel of the endoscope connector;
e) moving a first clip engagement member and a second clip engagement of the clip applying instrument to apply a force to first and second sides of the clip to move the clip from a closed position to an open position; and
f) releasing the clip to return to the closed position to clamp onto tissue.

In some embodiments, the clip is placed at an angle between about 45 degrees and about 90 degrees to the longitudinal axis of the endoscope. The endoscope can be advanced relative to the endoscope connector to view target tissue.

In some embodiments, advancing the flexible instrument guide distal of the first channel automatically causes the flexible guide to bend.

The flexible instrument guide can in some embodiments be advanced distal of the distalmost end of the scope and distal of the opening in the endoscope connector.

The method can further include in some embodiments the step of actuating an actuator operatively connected to the flexible instrument guide to bend the instrument guide.

The method can further include the step of advancing a dissector through a working channel of the endoscope.

The method can further include disengaging the first channel from the endoscope connector so bending of the channel is independent of articulation of the endoscope.

The method can further include the step of selectively linking or unlinking the instrument guide from the endoscope.

In accordance with another aspect of the present invention, a device for tensioning tissue during an endoscope tissue dissection procedure is provided, the device comprising a first clip configured to attach to tissue to be dissected, a second clip configured to attach to a wall of a body lumen, and a link connecting the first clip to the second clip. The first clip is attached to a first end of the link and the second clip is attached to the opposing end of the link.

In some embodiments, the link of the tension device comprises a suture or wire. In some embodiments, the link comprises a spring.

In some embodiments, the tension on the link can be adjusted in situ.

In some embodiments, the link extends through the second clip and through an endoscope for access at a proximal end to a user.

In some embodiments, the link extends adjacent an endoscope through a channel of an endoscope connector, e.g. a hub, attached to the endoscope.

In accordance with another aspect of the present invention, a method for detecting a loop in a colonoscope during insertion of the colonoscope through the colon is provided comprising the steps of:
a) attaching a sheath to a colonoscope, the sheath having a first channel to receive the colonoscope and a second channel to receive a sensor;
b) detecting a loop in the colonoscope during insertion; and
c) displaying the loop on a graphic display in communication with the sensor.

The method can further comprise the step of inserting a stiffening member into a channel of the endoscope to straighten the loop if detected.

In some embodiments, the sensor is built into a wall of the sheath. In some embodiments, a second sensor is provided, the second sensor communicating with the graphic display, wherein two images of the loop of the colonoscope are displayed on the graphic display.

In accordance with another aspect of the present invention, a system is provided comprising a shape detecting sensor positionable in a working channel of a colonoscope or connectable external of the colonoscope, the sensor communicating with an external reader to provide a graphic representation of a shape of the colonoscope

### BRIEF DESCRIPTION OF THE DRAWINGS

So that those having ordinary skill in the art to which the subject invention appertains will more readily understand how to make and use the surgical apparatus disclosed herein, preferred embodiments thereof will be described in detail hereinbelow with reference to the drawings, wherein:

FIG. 1 is a side view in partial cross-section of a first embodiment of the system of the present invention shown in use within the colon of a patient for a tissue resection procedure;

FIG. 2 is a close up view of the distal region of the system of FIG. 1;

FIG. 3 is a view similar to FIG. 1 (without showing the body) showing actuation of the link to curve the instrument receiving channel of the system;

FIG. 4 is a view similar to FIG. 3 showing an endoscopic grasper advanced through the instrument receiving channel;

FIG. 5 is a view similar to FIG. 4 showing within the colon a tissue stabilizer advanced through the instrument receiving channel of the system and a tissue dissector advanced through a working channel of the endoscope;

FIG. 6 is a close up view of the distal region of the system of FIG. 5 showing the stabilizer and grasper at the target tissue;

FIG. 7A is a close up view of the distal region of an alternate embodiment of the system of the present invention having two links for curving/bending the instrument receiving channel further away from the target tissue;

FIG. 7B is a close up view of the distal region of an alternate embodiment of the system of the present invention having a tensioning member in the form of a wire for curving/bending the instrument receiving channel, the instrument receiving channel shown in the unbent (linear) position;

FIG. 7C is a view similar to FIG. 7B showing the tensioning member in the form of a wire, tensioned to bend and rigidify the instrument receiving channel;

FIG. 7D is a close up view of the distal region of an alternate embodiment of the system of the present invention having a tensioning wire for curving/bending the instrument receiving channel and a plurality of wires bendable to form a tissue supporting structure, the instrument receiving channel shown in the bent position;

FIG. 7E is a view similar to FIG. 7D showing an alternate embodiment having a cover over the tissue supporting structure to prevent tissue invagination;

FIG. 8 is a side view in partial cross-section of an alternate embodiment of the system of the present invention having a joystick like single-hand controlled pivoting actuator for curving/bending the instrument receiving channel;

FIG. 9 is a transverse cross-sectional view of an alternate embodiment of the system of the present invention;

FIG. 10 is a cross-sectional view taken along line B-B of FIG. 9 showing the instrument receiving channel in the retracted position;

FIG. 11 is a cross-sectional view similar to FIG. 10 showing, within the colon, the instrument receiving channel in the advanced position with an endoscopic grasper advanced therethrough and the tissue stabilizer advanced from another instrument channel;

FIG. 12 is a cross-sectional view taken along line C-C of FIG. 9 showing the cable tensioned and endoscope locking member in the locked position (condition) to secure the hub/cap of the system to the endoscope;

FIG. 13 is a close up view of the system of FIG. 12 showing the cable not tensioned and the locking member in the disengaged (unlocked) position (condition) to unlock the hub/cap of the system to the endoscope;

FIG. 14 is a sectional view of an alternate embodiment of the system of the present invention wherein the hub has an articulation feature;

FIG. 15 is cross-sectional view of an alternate embodiment of the system of the present invention having a tubular liner extension within the endoscope channel of the hub/cap;

FIG. 16 is a transverse cross-sectional view taken along line D-D of FIG. 15;

FIG. 17 illustrates an endoscope inserted into the endoscope channel of the hub of the system of FIG. 15, the endoscope shown in the retracted position relative to the hub;

FIG. 18 is a view similar to FIG. 17 showing an instrument guide extending through the channel of the hub, the endoscope shown in the advanced position relative to the hub;

FIG. 19 is a view similar to FIG. 18 showing, within the colon, the instrument receiving channel (guide) advanced through the channel of the hub and an endoscopic grasper advanced through the instrument channel;

FIG. 20 is a side view in partial cross-section of an alternate embodiment of the system of the present invention having a spacer and a spring;

FIG. 21 is a view similar to FIG. 20 showing the instrument receiving channel advanced through the channel of the hub, an endoscopic instrument advanced through the instrument receiving channel, and the conduit advanced to compress the spring of the hub;

FIG. 22 is a cross-sectional view of an alternate embodiment of the present invention wherein the system and endoscope are connected to move together, the system shown with the instrument receiving channel advanced through the channel of the hub and an endoscopic instrument advanced through the instrument channel;

FIG. 23 is a cross-sectional view of an alternate embodiment of the system of the present invention having an inflatable balloon for stabilizing the system;

FIG. 24 is a cross-sectional view of an alternate embodiment of the present invention;

FIG. 25 is a close up view of the system of FIG. 24 showing rotation of the link to curve the instrument channel;

FIG. 26 illustrates the system of FIG. 24 mounted to an endoscope and positioned within the colon, and further showing the instrument receiving channel curved and an endoscopic grasper advanced through the instrument receiving channel;

FIG. 27 is a side view in partial cross-section of an alternate embodiment of the system of the present invention, shown in the colon, wherein a clip is delivered through a channel of the hub;

FIG. 28 is a view similar to FIG. 27 showing the clip applied to the tissue and an endoscopic dissecting snare placed around the tissue to be dissected;

FIG. 29 is a view similar to FIG. 28 showing a tissue stabilizer advanced through the channel of the hub;

FIG. 30 is a side view of an alternate embodiment of the system of the present invention having an instrument receiving channel linked to the hub by a flexible member, the flexible member shown in the untensioned position;

FIG. 31 is a view similar to FIG. 30 with the endoscope removed and the system shown outside the colon;

FIG. 32 is close up view of the system of FIG. 30 showing the flexible member tensioned to link the endoscope and instrument receiving channel;

FIG. 33 is a view similar to FIG. 32 showing the flexible member untensioned to operably separate the endoscope and instrument receiving channel;

FIG. 34 is a side view of an alternate embodiment of the system of the present invention having a control handle for operating the instrument guide and endoscopic instrument independently;

FIG. 35 is a close up view of the control handle of the system of FIG. 34;

FIG. 36 is a side view of an alternate embodiment of the system of the present invention having a collapsible instrument receiving channel, the system shown in the colon with an endoscopic grasper and snare at the target tissue;

FIG. 37 is a close up view of the system of FIG. 36 showing the instrument receiving channel in the collapsed position (condition) (outside the colon);

FIG. 38 is a transverse cross-sectional view taken along line A-A of FIG. 37;

FIG. 39 is a transverse cross-sectional view taken along line B-B of FIG. 37;

FIG. 40 is a view similar to FIG. 37 showing the instrument channel bent along with the articulation (bending) of the endoscope;

FIG. 41 is a view similar to FIG. 37 showing an instrument guide partially inserted through the instrument receiving channel;

FIG. 42 is a view similar to FIG. 37 showing the instrument guide advanced out of the distal opening of the instrument receiving channel;

FIG. 43 is a transverse cross-sectional view taken along line B-B of FIG. 41;

FIG. 44 is a view similar to FIG. 42 showing the instrument channel with an inserted endoscopic grasping instrument bent along with the articulation (bending) of the endoscope and an endoscopic resecting device inserted into the working channel of the endoscope;

FIG. 45 is a side view of an alternate embodiment of the system of the present invention having a collapsible instrument receiving channel with axially spaced openings;

FIG. 46A is a view similar to FIG. 45 showing the instrument guide partially advanced through the more proximal opening of the instrument channel;

FIG. 46B is a view similar to FIG. 46 showing the instrument guide advanced out of the distal opening of the instrument channel;

FIG. 46C is a view similar to FIG. 46 showing articulation of the endoscope independent of the instrument guide and a grasping instrument extending through a working channel of the endoscope;

FIG. 47 is a side view in partial cross-section of an alternate embodiment of the system of the present invention shown in use within the colon of a patient for a tissue resection procedure and showing a tissue grasper inserted through a working channel of the endoscope and a tissue dissector inserted through the instrument guide external of the endoscope;

FIG. 48 is a side view in partial cross-section of an alternate embodiment of the system of the present invention shown in use within the colon of a patient for a tissue resection procedure and showing a tissue grasper inserted through a working channel of the endoscope and another tissue grasper inserted through the instrument guide external of the endoscope;

FIG. 49 is a side view of an endoscope inserted into the colon and a dissector advanced through a channel of the endoscope for dissection of a lesion in the colon;

FIG. 50 is a side view of one embodiment of a tissue tension device of the present invention for providing tissue traction;

FIG. 51 is a side view of an alternate embodiment of a tissue tension device of the present invention;

FIG. 52 is a side view of an alternate embodiment of a tissue tension device of the present invention;

FIG. 53 is a side view of an alternate embodiment of a tissue tension device of the present invention wherein the clips are connected by an extension spring;

FIG. 54 is a side view of an alternate embodiment of a tissue tension device of the present invention wherein the clips are connected by a loop;

FIG. 55 is a side view of an alternate embodiment of a tissue tension device of the present invention wherein the link is adjustable to adjust the distance between the two clips;

FIG. 56 illustrates the tension device of FIG. 50 wherein the lesion clip is attached to the lesion and the anchor clip is attached to the colon wall;

FIG. 57 is a view similar to FIG. 56 show partial dissection of the lesion and loosening of the tension of the link;

FIG. 58 is a view similar to FIG. 57 showing repositioning of the tissue tension device to maintain tension on the link;

FIG. 59 illustrates the tension device of FIG. 55 wherein the lesion clip is attached to the lesion and the anchor clip is attached to the colon wall

FIG. 60 is a view similar to FIG. 59 showing partial dissection of the lesion with the adjustable link tensioned;

FIG. 61 illustrates a tool for tensioning the link of the tissue tension device of FIG. 59;

FIG. 62 is a view similar to FIG. 61 showing the tool retracted to reduce the length of the link to tension the link;

FIG. 63 is a side view of an alternate embodiment of a tissue tension device of the present invention showing the lesion clip attached to the lesion and the anchor clip attached to the colon wall, and further showing the tensioning link extending through the working channel of the endoscope for adjusting/tensioning from the proximal end of the link;

FIG. 64 is a side view of an alternate embodiment of a tissue tension device of the present invention showing the lesion clip attached to the lesion and the other end of the link extending through a channel of the hub;

FIG. 65 illustrates the use of two tissue tension devices—the devices of FIG. 50 and FIG. 55;

FIG. 66 illustrates an alternate embodiment of the present invention having a link with four clips;

FIG. 67 illustrates a colonoscope inserted into a colon of a patient;

FIG. 68 shows the colonoscope inserted into the sigmoid portion of the colon wherein the sigmoid portion is deformed by the colonoscope loop;

FIG. 69 illustrates a system for sensing and displaying a shape of the colonoscope to detect loops;

FIG. 70 illustrates the endoscope having a sensor of the present invention inserted into the colon and the image displayed on a screen;

FIGS. 71 to 74 illustrate embodiments of the present invention wherein the shape sensor is not directly integrated into the colonoscope but is a standalone device added to the scope wherein:

FIG. 71 is a cross-sectional view of one embodiment of the sensor device of the present invention showing the sensor integrated into the wall of the sensor device;

FIG. 72 illustrates the sensor device of FIG. 71 positioned in a working channel of the colonoscope;

FIG. 73 illustrates an alternate embodiment wherein the sensor is integrated into a sheath which is positioned over the colonoscope;

FIG. 74 illustrates an alternate embodiment wherein the sensor is inserted into a sheath which is positioned over the colonoscope;

FIG. 75 is a transverse cross-sectional view of an alternate embodiment of the present invention having a first sensor inserted into the working channel of the colonoscope and a second sensor inserted into a lumen of the sheath placed over the colonoscope, the second sensor positioned externally of the endoscope;

FIG. 76 illustrates two images of the loops, detected by the two sensors of Figure graphically displayed on a screen;

FIG. 77 is a side view of a stiffening device of the present invention for straightening the colonoscope; and

FIG. 78 is a cross-sectional view showing the stiffening device within a working channel of the colonoscope.

### DETAILED DESCRIPTION OF PREFERRED EMBODIMENTS

The present invention provides systems and methods to aid tissue dissection in hollow organs, such as a colon, small intestine, esophagus, stomach, etc.

When performing a resection, a surgeon typically manipulates tissue with two instruments: 1) a cutting device, such as a scalpel, scissors, or an electrosurgical device; and 2) a retractor that allows the surgeon to tension tissue that is being cut by the cutting device since tissue is easier to cut, when under tension. Pulling tissue away from the cutting point makes tissue cutting safer allowing the surgeon to visualize the area of resection and a cutting plane.

During endoscopic mucosal resection (EMR), the physician dissects mucosal tissue superficially, while during endoscopic submucosal dissection (ESD), the dissection penetrates deeper layers of the wall and during endoscopic full-thickness resection (EFTR) procedure allows the physician to go through the wall completely. These procedures are performed endoscopically (minimally invasively) via the body lumens, for example, a colon with a trans-anal approach or an esophagus and a stomach trans-orally. A stable and reliable tissue traction is beneficial as it facilitates cutting. Dynamic retraction with continuously adjustable tissue tension is also beneficial.

The systems of the present invention are in preferred embodiments utilized for endoscopic resection and for tissue manipulation and retraction during endoscopic procedures in the upper gastrointestinal tract, for example, in the esophagus, stomach, small intestine, including duodenum, and in the lower gastrointestinal tract, for example, in the rectum, sigmoid, colon, and appendix. Systems of the present invention have a passive navigation and rely on the flexible endoscope for insertion into the body lumen and reaching the target site. Flexible endoscopes, such as a colonoscope, commonly have an articulating mechanism located in their distal portion to facilitate navigation of the scopes through torturous anatomical structures. The articulating mechanism is typically 2 to 4 inches long and remotely controlled by users from the proximal end of the endoscope.

The endoscope and system of the present invention can be inserted simultaneously. Alternatively, the scope could be advanced first, then the system advanced by using the scope as a guide.

In some uses/procedures, when the scope and the system reach the vicinity of the target site, the system further relies on the scope's articulation mechanism to guide it directly at the target tissue. In such embodiments, it could be beneficial for the system and the scope to be interconnected at the distal end of the scope, such that the entire length of the scope's articulating mechanism is guiding the system to the target site.

In other uses/procedures, the precise guidance by the scope's articulation mechanism is not required, and the scope could be used for the system insertion and delivery only. In such embodiments, it could be beneficial for the system and the scope to be interconnected only proximally to the articulating mechanism of the scope. Alternatively, they could be interconnected at the scope's articulating mechanism, but proximally to the scope's distal end.

In other uses/procedures, the system relies on the scope for delivery and guidance to the target tissue, but when the system is in position and engages with the target tissue (for example, when the system includes a grasper, and the grasper grabs the target tissue), it no longer needs guidance from the articulating mechanism and can maintain its position. In such embodiments, it could be beneficial for the scope and the system to be interconnected at or near the distal end during the delivery and guidance, but preferably disconnectable, so that the distal end of the scope could move independently from the system. The independent movement of the distal end of the scope would allow the user to manipulate an endoscopic device that could be inserted via the instrument channel of the scope, while the system will not be affected by such movement. The scope and the system could remain interconnected proximally to the articulating mechanism of the scope or at the scope's articulating mechanism, but proximally to the scope's distal end, such that the scope still provides support for the system.

The systems of the present invention in some embodiments include multiple elements, such as an external channel and an instrument guide, which are described below in detail. The external channel and the instrument guide could be deployed/inserted into a body lumen simultaneously, i.e. the instrument guide is inserted into the external channel before both are inserted into the body lumen. Alternatively, the external channel could be deployed/inserted into the body channel first, then, when it reaches a target tissue, the instrument guide is inserted into the channel. In some instances, it might be beneficial to partially insert the external channel, then begin inserting the instrument guide before the external channel reaches the target tissue. After that, they both could be advanced together or continue to be inserted incrementally (a partial advancement of the external channel followed by a partial advancement of the instrument guide) until both reach a desired position relative to the target tissue site. Other embodiments have an external channel forming the instrument guide.

These various methods of the system insertion are relevant because of the differences in the mechanical properties of the external channel and the instrument guide. In some embodiments utilizing a flexible instrument guide with the instrument receiving channel, the design of the external channel has limited or no effect on the ability of the endoscope to articulate and advance through a potentially torturous and small size anatomy of human lumens, such as a colon, an esophagus, etc. This means that the external channel 1) has a relatively small cross-sectional profile at least at/near its distal end, 2) is soft/flexible to allow the scope to fully articulate, yet 3) provides support structure for the instrument guide when one is being inserted into the external instrument channel. Preferred embodiments of the instrument guide are 1) pushable, so that it could be inserted from the proximal end to the distal end of the external instrument channel; 2) torqueable to control the rotational orientation of its distal tip, such that it could guide a flexible endoscopic instrument, such as a grasper, to the target tissue; and 3) have sufficient structure to support the flexible endoscopic instrument during tissue manipulation even when the instrument guide is advanced distally to the external channel. Therefore, in some cases, it might be beneficial to have the external channel advanced first to take advantage of its smaller profile and flexibility, before the stiffer instrument guide is advanced.

As noted above, the systems of the present invention in some embodiments is utilized for endoscopic resection and provides for tissue manipulation and retraction during endoscopic procedures in the upper gastrointestinal tract, for example, the esophagus, stomach, duodenum, and in the lower gastrointestinal tract, for example, the rectum, sigmoid colon, appendix. The system in general includes a) an endoscope connector which in some embodiments includes a hub or cap (or sheath) attachable to or placeable over an endoscope and b) an instrument receiving channel (or alternatively multiple instrument receiving channels) attached thereto which in some embodiments forms a passageway for a flexible instrument guide. The cap or hub (or sheath or other endoscope connector) also includes another channel (or alternatively multiple channels) for passage of an endoscopic instrument such as an instrument to stabilize the tissue, e.g., lesion being dissected. The channels are outside, i.e., external of, the endoscope. The instrument channel (or instrument guide in embodiments where provided) guide is configured to direct an endoscopic instrument toward the target site at an increased angle as opposed to a more direct angle, i.e., in line relative to the scope, if the instrument is inserted through a channel of the endoscope. Various embodiments of the endoscope connectors and instrument channels and guides are discussed below. Note that the hubs/caps/sheaths (or other endoscope connectors) of the various embodiments disclosed herein can include one or more of the channels for passage of endoscopic instruments and/or one or more of the flexible instrument guides.

The system and method of the present invention in some embodiments provides for movement, e.g., bending, of the instrument channel or instrument guide independent of bending of the scope. In some embodiments, the user can selectively decide whether to have the instrument channel or instrument guide articulate along with the scope, i.e., tie together the movement of the endoscope and instrument channel/guide, or separate the movements of the endoscope and instrument channel/guide so that articulation of the endoscope does not affect articulation of the instrument channel/guide and bending of the instrument channel/guide can be performed independent of the endoscope position. Various embodiments to achieve this are discussed in detail below.

The systems of the present invention can be retrofitted to a conventional endoscope if desired. The endoscopes can be articulated by wires or other structures currently being utilized in the conventional scopes.

The systems of the present invention can also be retrofitted to a robot, e.g., a robotic arm, similar to that currently being utilized for endoluminal surgery. In this manner, one or more of the instrument channels, instrument guides, instruments. actuators, etc. could be remotely robotically controlled. Thus, bending, axial movement, rotation, etc. of the components of the system can be effected via robotics, e.g., by a user at a remote console.

It is understood that multiple systems described below could be used simultaneously in a single surgical procedure.

It is also understood that features described for one of the embodiments could also be beneficial and utilized for other embodiments.

The present invention disclosed herein can also include a tissue tensioning device that applies tension to the lesion with the body lumen, e.g., colon, being dissected. Various embodiments of tissue tension devices are discussed in detail below which include non-stretchable devices as well as adjustable devices which can adjust the tissue tension, including adjustments in situ.

The present invention in some embodiments can also include a sensor that facilitates insertion of the endoscope, e.g., a colonoscope. In this aspect of the present invention, a system and method are provided wherein the sensor can detect an undesired loop in the endoscope during insertion and communicate with a monitor to graphically display the loop to alert the clinician so appropriate action can be taken in diagnostics or to straighten the loop. Thus, the sensor alerts the user that the loop is forming and helps the user to insert the scope completely or to the target site.

Note the tissue tension device and/or the sensor can be used in conjunction with the systems and methods of the present invention utilizing instrument guides to access the target tissue or alternatively used independently of the other inventions disclosed herein.

In the drawings of the present application, like reference numerals identify similar structural features of the devices disclosed herein.

As used herein, the term “proximal” refers to the portion, region or component closer to the user and the term “distal” refers to the portion, region or component further from the user.

### Systems for Endoscopic Resections

Turning first to the systems for endoscopic resections, these systems provide flexible instrument guides to increase the access angle of endoscopic instruments to the target tissue, such as a lesion in the colon. The instrument channels or guides can have a preset curve to automatically bend when exposed or alternatively be actively bent via an actuator. The instrument channels or guides are part of a hub or cap (or sheath or other endoscope connector) that is attached to a distal end of an endoscope, or separate components connected to the hub or cap (or sheath or other endoscope connector), and can in some embodiments be attached to a conventional endoscope. Attachment to robotic arms for robotic control is also contemplated. As will be described below, the instrument guides can be the instrument receiving channel itself or a separate guide placed within the instrument receiving channel.

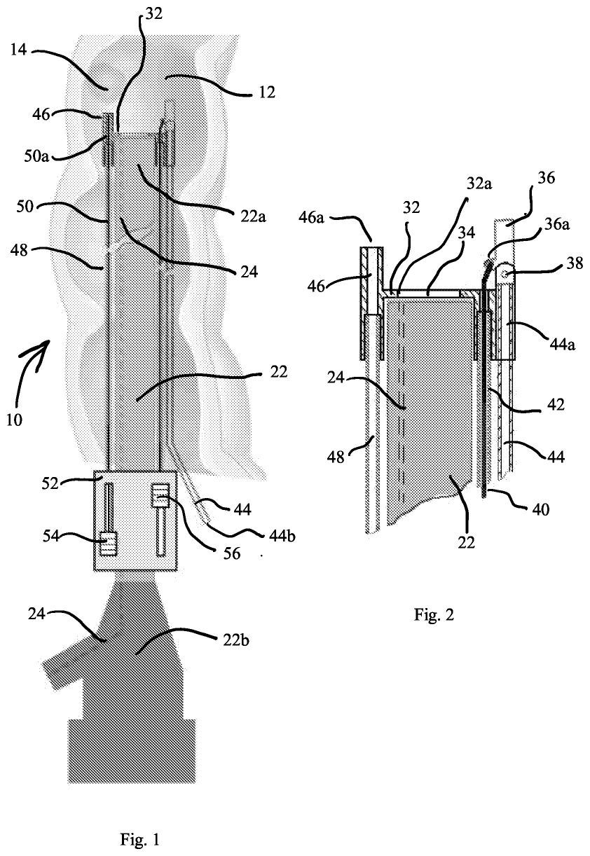

Turning first to the embodiment of FIGS. 1 and 2, a system using an active control of the bending of the instrument channel which forms an instrument guide is disclosed. The system is designated generally by reference numeral 10, and is used for endoscopic tissue resection procedures, such as mucosal resection and submucosal dissection, although it can also be used for other procedures. The system 10 is inserted inside of a body lumen 12, for example an esophagus or a colon, until it reaches the target tissue 14, such as a lesion, perforation, diverticula, bleeding ulceration, etc.

The system 10 includes an endoscope connector in the form of a cap 32 that is removably attached to the distal end 22a of a flexible endoscope 22. As shown, the cap terminates proximally at a distal region of the endoscope 22. Alternatively, the system could be implemented as a sleeve or a sheath that is placed over the flexible endoscope 22 and can extend over a partial, substantial or entire length of the endoscope rather than just a distal region as with cap 32. That is, the sleeve would have a length longer than the cap 32 and have a distal end that in the working position is placed near the distal end 22a of the scope 22 and a proximal end extending therefrom which can in some embodiments extend outside of the patient's body close to where a control mechanism 52 (described below) is located. All concepts of the cap and hub that are described below could also be implemented as a sleeve/sheath. Thus, the discussion of the components of the cap (and hub) system, including its alternative versions, are fully applicable to the sleeve/sheath system.

Although the embodiments of the cap have a wall that prevents the endoscope from extending past the distal edge of the cap and the embodiments of the hub have a distal opening through which the endoscope can pass through, the cap can alternatively have a distal opening to enable passage of the endoscope and the hub can alternatively have a wall that prevents the endoscope from passing therethrough. Therefore, the terms “cap” and “hub” are used interchangeably herein. It should also be appreciated that the cap or hub are examples of the endoscope connectors of the present invention as other endoscope connectors are also contemplated and features and functions of the embodiments described with hubs or cap are also applicable to embodiments having other endoscope connectors.

Also note that in some embodiments, the instrument (tool) receiving channel of the endoscope connector, e.g., hub or cap, directly receives the endoscopic instrument and therefore the instrument receiving channel itself forms the instrument guide. Some of these embodiments for example have a pivotable linkage to angle the instrument receiving channel. In other embodiments, the instrument receiving channel of the endoscope connector, e.g., hub or sheath, receives an instrument guide which receives the endoscopic instrument. Stated another way, in the latter, the instrument guide also functions as the instrument receiving channel and thus can also be considered an instrument receiving channel. Either way, the instrument receiving channel receives the endoscopic instrument (either directly or by an intermediate instrument guide). The component that directly receives the endoscopic instrument (e.g., the instrument receiving channel or instrument guide if received in the channel(, is bendable toward the longitudinal axis of the endoscope connector or endoscope to direct the endoscopic instrument at an angle to the longitudinal axis of the scope or endoscope connector.

In some embodiments the instrument channel has a linkage or steerable wire and the instrument guide has a linkage or steerable wire so that gross adjustment can be made with the instrument channel and fine adjustments can be made with the instrument guide. In some embodiments, the instrument itself can be articulated. In this manner, in these embodiments, all three elements are maneuverable and each of the components can be controlled separately for axial, lateral and rotational movement.

In some embodiments, instead of pulling on the wire to deform the end, a grasper can be inserted through the working channel of the endoscope or a channel of the hub or other connector and used to pull on the end of channel to deform it and hold it in place.

Referring back to FIGS. 1 and 2, the cap 32 has an aperture 34 to allow for visualization and passage of endoscopic instruments via the tool (working) channel 24 of the endoscope 22. The cap 32 can have a wall 32a which limits distal movement of the endoscope 22 beyond the aperture 34 of the cap 32. The cap 32 has an instrument port or conduit 46 and a flexible instrument channel 48 that extends proximally therefrom. One or both of the port 46 and channel 48 can be monolithic with the cap 32 or separate attached members. In the illustrated embodiment, the channel 48 is positioned within and attached to port 46. The channel 48 extends proximally from the port 46 to control handle 52. Control handle 52 could be completely unattached or optionally attached to the shaft of the endoscope scope or be kept stationary by fixation to a stationary unit, such as an operating table with or without a bracket.

During use, an instrument 50 extends through channel 48 such that its distal end extends within the instrument port 46 and exits out of distal opening 46a in port 46. The instrument 50 is controlled by the actuator, e.g., slider 54, on control handle 52, which is shown in the retracted position in FIG. 1. Sliding the slider 54 distally advances the instrument 50 through the channel 48 and port 46; sliding the slider proximally retracts the instrument 50 in the port 46 or channel 48. Note the port 46 and channel 48 are shown as separate components but alternatively can be monolithic.

Attached to the cap 32 at pivot point 38 is an articulating link 36 that is rotated remotely relative to the pivot point 38 attached to extension 37 by manipulating the control mechanism 56 on control handle 52 which is outside the patient's body. The link 36 has an eyelet 36a to receive a pull cable 40. Pull cable 40 is located within the channel 42 of cap 32 which extends external of the scope 22 and is radially spaced therefrom. The distal end of the pull cable 40 extends beyond the distal edge of the cap 32 and out the distal opening of channel 42 to attach to the link 36. The proximal end of the cable 40 is attached to a slide or actuator 56 at the control mechanism 52.

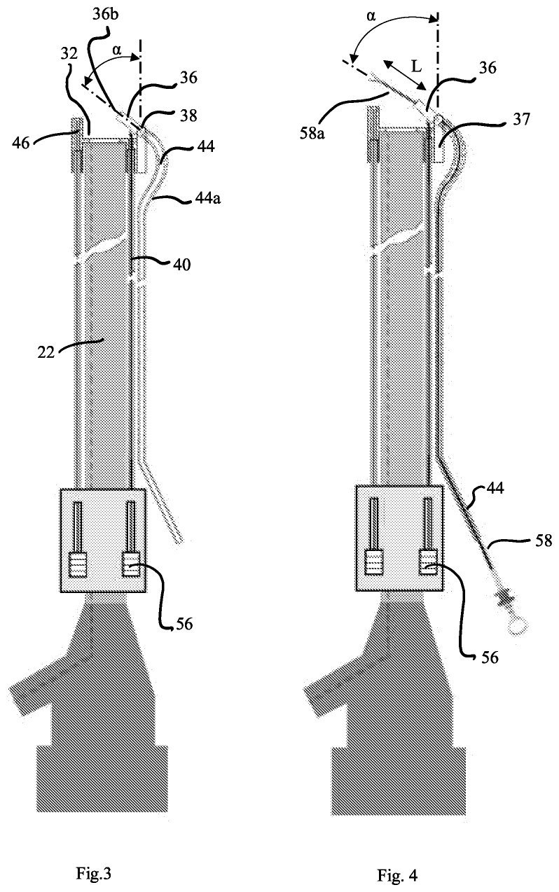

When the slide 56 is retracted from its distal position of FIG. 1 to its proximal position of FIG. 3, pull cable 40 pulls the link 36 at the attachment point 36a and rotates it about pivot point 38 from its axial or substantially axial position of FIG. 1 to its angled position of FIG. 3. The distal portion 36b of the link 36 is thus oriented inwardly toward the longitudinal axis of the cap 32 and the longitudinal axis of the scope 22, and generally toward the port 46, which is located on the opposite side of the cap 32. By controlling the travel distance of the slide 56, the articulating angle α of the link 36 could be adjusted, as necessary, i.e., further proximal movement of the slide 56 increases the angle of the link 36 with respect to the longitudinal axis of the scope 22.

A flexible instrument receiving channel (guide) 44 is attached at is distal end to the link 36 by various methods such as glued, crimped, welded, etc. When the link 36 rotates, it deforms the flexible instrument channel 44.

In the initial position, the distal portion 44a of the flexible instrument channel 44 is generally aligned with (generally parallel to) the cap 32 as shown in FIG. 1; in the rotated position of link 36, the distal portion 44a of the flexible instrument channel 44 is curved as shown in FIG. 3. In the illustrated embodiment, it curves in a S-shape, however, other curved (non-linear) configurations such as a C-shape, L-shape or J-shape are also contemplated. The flexible instrument channel 44 serves as a conduit for a flexible endoscopic instrument such as a flexible endoscopic grasper, a snare, an electrosurgical knife (such as Olympus DualKnife™ electrosurgical knife), an endoscopic clip (such as Boston Scientific's Resolution™ Clip) or other flexible endoscopy accessories. The stabilizing instrument 50 described below could be a standalone endoscopic device and could also be inserted through the channel. The endoscopic instruments are advanced out the distal opening of channel 44 and extend distal of the distal end of the scope 22. The proximal end 44b of the instrument channel 44 extends to or near the proximal end of the system 10, and in some embodiments outside the patient, for access by the user so the user can insert the endoscopic instrument through the proximal opening in proximal end 44b and advance it through instrument channel 44. Thus, as can be appreciated, the instrument channel 44 and instrument channel 48 are external of the scope 22 as they can for example extend alongside the scope 22. The external positioning can in some embodiments be radially spaced from the scope.

FIG. 4 shows by way of example a flexible endoscopic grasper 58 inserted into the flexible instrument channel 44 such that its distal end 58a is positioned at an angle α relative to the axes of the scope 22 and the cap 32 due to the pivoting of the linkage 36. By sliding the flexible endoscopic grasper 58 in and out relative to the flexible instrument channel 44, users can control the length L. The length L is controlled independently from the control of the articulating angle α (the articulating angle is controlled by the extent of slider 56 movement which effects the extent of axial movement of the cable 40 and the angle of the linkage 36). Note that changes to the length L results in tissue pushing towards and pulling away from the wall, while changes of the angle α creates a side pull. Note the instrument channel 44 can be moved to its curved configuration of FIG. 4 either before or after the endoscopic instrument is inserted therethrough.

To enhance stability and improve force transfer from the linkage 36, the distal end 58a of the flexible endoscopic grasper 58 could be reinforced/made stiffer that the proximal section of the flexible endoscopic grasper 58.

While only one articulating link 36 with a flexible instrument channel 44 and one instrument port 46 with a flexible instrument channel 48 are shown, multiple links, ports and instrument channels are also contemplated. Also, the articulating link 36 provides one example of a mechanism to actively bend/articulate the instrument channel 44 as other actuable mechanisms could also be provided.

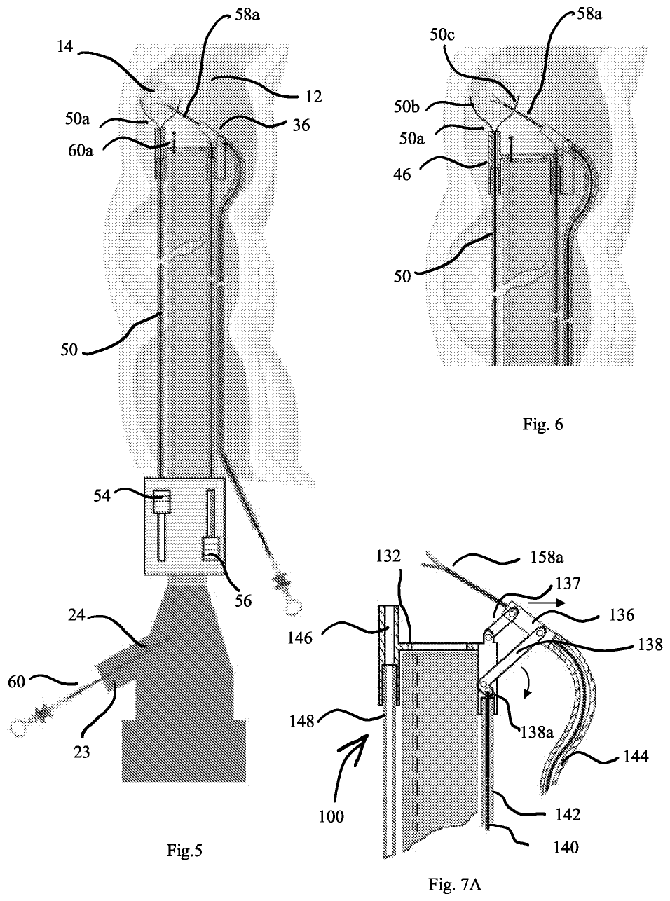

As discussed above, instrument channel 50 with port 46 are configured to receive another endoscopic instrument. By advancing a slide 54 (FIGS. 5 and 6), the distal end 50a of a flexible instrument 50 is advanced out of the instrument port 46 allowing the two arms and 50c of the instrument 50 to flex out forming a “fork-shaped” tissue stabilization structure. The arms 50b and 50c could be made from a resilient material, for example Nitinol alloy. When the target tissue 14 is lifted using the flexible grasper 58, the flexible instrument 50 creates tissue stabilization and tension/counter tension. A cutting instrument such an electrosurgical knife 60 (e.g. an Olympus DualKnife™ electrosurgical knife) is introduced via the scope's tool channel (working channel) 24, and the working end 60a of the knife 60 is used for dissection of the target tissue 14. Thus, using the instrumentation shown in FIGS. 5 and 6, a) the grasper 58 is angled toward the target tissue 14 at an increased angle to grab tissue due to the articulation of the link 36; b) the instrument 50, exiting on an opposing side of the cap 32 (and scope 22) stabilizes the tissue; while c) the instrument 60 going through the scope channel 24 dissects the tissue. The instruments 58 and 50 are controlled by the sliders 56, 54 of the system 10 (or by other types of actuators) while the instrument 60 is controlled at its proximal end which extends through a side port 23 of endoscope 22, or alternatively can extend through a proximal opening in the endoscope 22.

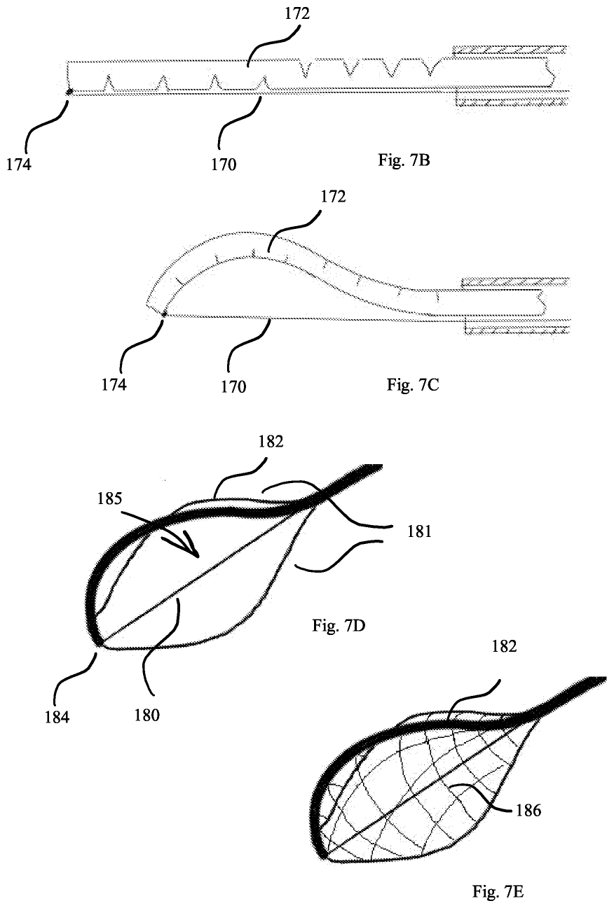

FIG. 7A illustrates an alternative system 100 of the present invention having multiple links. In this embodiment, the articulating mechanism 138 of cap 132 is in the form of a link and is attached at one end 138a (the proximal end) to pull cable 140. The link 138 is activated by the pull cable 140, which extends though conduit (channel) 142 in a similar manner as cable 40 of FIG. 1. The articulating mechanism 138 is attached at the other end to linkage 136. A second link 137 extends from an eyelet in cap 132 to a distal region of linkage 136. When the cable 140 is pulled within the conduit 142, the articulating mechanism (link) 138 rotates the articulating link 136 and moves it in a lateral direction outwardly from the cap 132, pivoting about pivot pint 138a, thereby increasing the distance of the distal opening of the instrument receiving channel 144 from the target tissue as it bends the instrument channel 144. This provides for increased working space for tissue manipulation as it allows linkage 136 to move further laterally away from the target tissue.

Note such rotation also pivots link 137 in the same direction. Cap 132 also includes port 146 and channel 148, functioning like port 46 and channel 48.

In some embodiments, linkages can be used to pivot one or both of the instrument (tool) receiving channel and the instrument guide or one or both of the instrument (tool) receiving channel and instrument guide can have preset shapes/curves as described below. In some embodiments, the endoscopic instruments have a feature capable of forcing the instrument receiving channel and/or instrument guide to take shape. either by the instrument having a pre-set non-linear shape or by articulation of the instrument, e.g., by an articulation mechanism such as steerable wires or cables. Note the instrument receiving channel and instrument guides could also have articulation mechanisms such as cables or wire for articulation of the distal portion

FIGS. 7B and 7C illustrate an alternate system having a tensioning member such as a pull wire directly attached to a distal end of the instrument receiving channel rather than a linkage for bending the channel. As shown, pull wire 170 is attached to instrument receiving channel 172 at attachment point 174. When the pull wire 170 is untensioned as shown in FIG. 7B, the instrument receiving channel 172 is in a linear or substantially linear position. The instrument receiving channel 172 can have cutouts, which can be formed by a laser cut metal tube. With the cutouts, when the instrument receiving channel 172 is untensioned, it is flexible. On the other hand, when tensioned, the wedges collapse to butt up against each other (closing the cutouts) and the channel 172 becomes rigid under tension. Thus, in the tensioned state, it becomes a load carrying member and becomes like a solid to maintain the angle rigid. When the wire 170 is tensioned, e.g., pulled by rotational or axial movement of an actuator, the pull wire 170 is tensioned to bend the instrument channel as shown in FIG. 7C.

In alternate embodiments, the tensioning member could be an instrument that is introduced via a working channel of the endoscope or via another conduit such as a conduit in the endoscope connector. For example, the instrument could be a grasper and the user can insert the grasper and use the grasper to grab the end of the instrument receiving channel and pull on it to bend the channel.

The system in some embodiments can include one or more wires 181 made of shape memory material such as Nitinol, although other materials are also contemplated. The wires 181 are also attached to the distal end of the instrument receiving channel 180 at or adjacent the pull wire attachment point 184 as shown in FIG. 7D. These wires 181 are bent when pull wire 180 is tensioned to bend instrument receiving channel 182. Bending of the wires 181 forms a tissue supporting structure creating a defined area. In some embodiments, the tissue supporting structure can expand to create an enlarged/expanded area/region 185. The tissue supporting structure keeps tissue away from the target or treatment region. For example, the tissue supporting structure can operate and keep the opposite wall of the colon away from the target area/region 185. The tissue supporting structure 185 in some embodiments can be covered by a cover 186 to enclose the tissue supporting structure to prevent entry of tissue as shown in the embodiment of FIG. 7E. The tissue supporting structure 185 in some embodiments can provide an expanded work space within the colon for maneuverability and use of the endoscopic instruments inserted through the instrument receiving channels and the endoscope.

It should be appreciated that the tissue supporting structure, with or without the cover, can be utilized with the other embodiments disclosed herein.

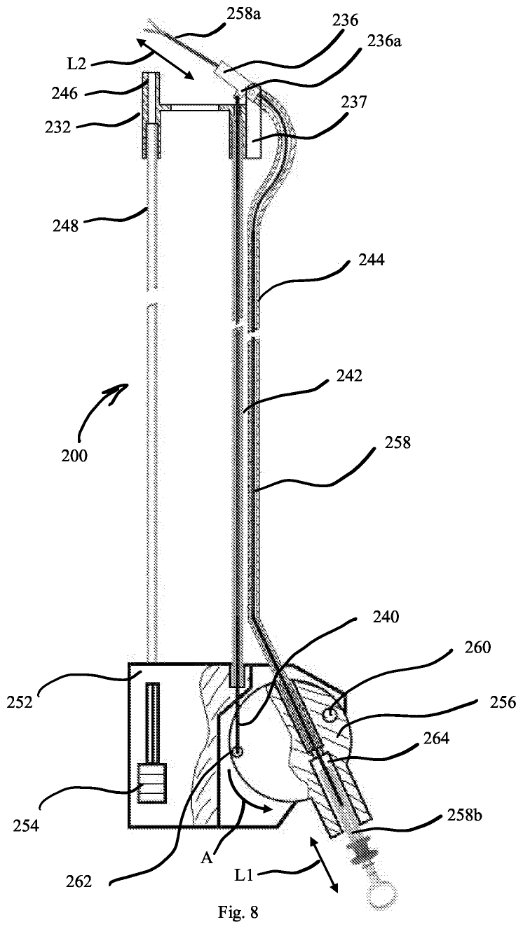

In the embodiment of FIG. 1, the sliders 54 and 56 are positioned in the control handle 52 which is aligned with the longitudinal axis of the cap 32 and the sliders 54 and 56 move axially. In the alternative embodiment of FIG. 8, the control handle 256 is offset from the longitudinal axis and the cable is actuated by a rotational motion.

More specifically, the system 200 of FIG. 8 has a control mechanism (control handle) 252 with a slider 254, similar to slider 54 of FIG. 1, for controlling axial movement of the instrument 50 through channel 248 and port 246 and an eccentric handle 256 for actuating the articulating link 236. A pull cable 240, like cable 40, is located within a channel 242 like channel 42 of FIG. 1, and is attached at one end to eccentric handle 256 at attachment point 262 and at the other end to link 236. Support (extension) 237 which is connected to link 236 at connection point 236a. Eccentric handle 256 rotates around the pivot 260 by an angle A to pull on the pull cable 240 to rotate the link 236 relative to the cap 232 to bend (articulate) the distal region of the instrument receiving channel 244 to the curved or bent configuration as shown. The handle 256 thus provides a joystick-like single-hand controlled pivoting actuator for curving/bending the instrument receiving channel.

An endoscopic instrument 258, such as a grasper, is inserted into instrument receiving channel 244. The endoscopic instrument 258 has a handle 258b that is located within a channel 264 of the eccentric handle 256. By moving the handle 258b axially within channel 264, a user can advance and retract the instrument's distal end 258a relative to the articulating link 236. By moving the handle 256 rotationally relative to the pivot 260 (clockwise or counterclockwise), a user can rotate the eccentric handle 256, and as a result, articulate the articulating link 236 (due to the pulling of cable 240). Therefore, a user can simultaneously control the endoscopic instrument 258 and the articulating link 236 with one hand.

FIGS. 9-13 illustrate an alternative system 300 of the present invention. Instead of a cap that could be mounted at the end of an endoscope, the system has a distal hub 332 with a channel 334 that is sized to allow an endoscope 322 to pass through the channel 334 and out distal opening (window) 334a. This allows the system and the scope to move independently of each other. The hub 332 includes a slot or channel 338 sized to receive a flexible tubular member 336 preset to a curved configuration and forming an instrument guide or instrument receiving channel.

The scope channel 334 can be positioned concentric to the hub 332 or alternatively could be positioned eccentrically relative to the hub 332, as shown in the cross-sectional view of FIG. 9 where it is offset from the longitudinal axis of the hub 332. Other features, such as an instrument port 346, cable attachment point 347, slots 338 and 339 could be located peripherally as shown. Other arrangements/positions of the channels are also contemplated.

The distal hub 332 includes an instrument port 346 on an opposing side of the hub 332 from the slot 338. Thus, like the slot 338 for the instrument guide 336, the instrument port 346 is spaced external of the scope 322. The instrument port 346 extends distally from instrument channel 348.

The tubular member 336 is connected with a flexible instrument receiving channel 344, extending proximally from the tubular member 344, forming a continuous path for a flexible endoscopic instrument 358, for example a grasper. Thus, the channel 344 also forms part of an instrument guide or instrument receiving channel. The flexible member 336 could be made out of a resilient material, such as Nitinol alloy, and is elongated and in the form of a tubular member. Alternatively, both link 336 and the channel 344 could be made out of a braided tubing to improve pushability. In another alternative, the link 336 and the channel 344 could be a single component with a continuous path for a flexible instrument 358.

The endoscopic instrument 358 moves relative to the slot 364 in handle 356 such that a) by moving the instrument 358 a distance L5 allows its distal end 358a to move in and out relative to the distal end 336b of the guide 336 by a distance L6 (L6 in preferred embodiments being the same as L5; and b) by rotating the instrument 358 by an angle β3, its distal end 358a rotates by an angle β4 (in preferred embodiments, β3 being the same as β4).

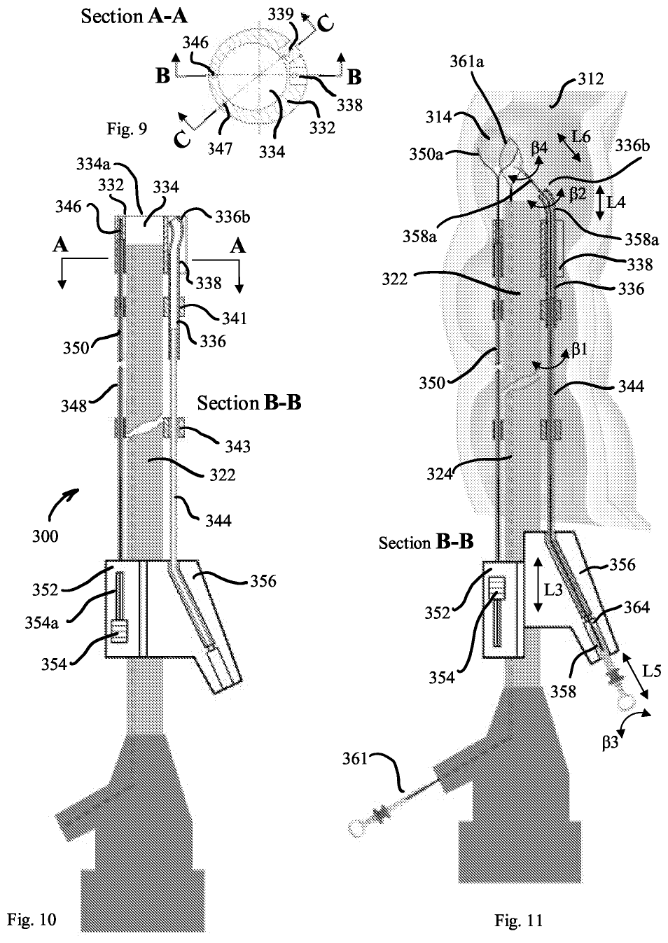

The slot 338 in the hub 332 allows the distal portion 336b of the instrument guide 336 to slide relative to hub 332 while minimizing the overall size of the system 300. It is contemplated that the instrument guide 336 and the channel 344 have sufficient structural rigidity, such as a column strength and torqueability, such that a) by moving the slider 356 of the control handle 352 by a distance L3, the distal end 336b of the guide 336 advances by a distance L4 relative to the hub 332; and b) by rotating the channel 344 by an angle β1, the distal end 336b rotates by an angle β2. The retracted position of the guide 336 is shown in FIG. 10 and the extended/exposed position of the guide is shown in FIG. 11. In the extended position, the guide 336 moves to its preset (or shape memorized) bent position to angle toward the lesion as shown. The system of FIGS. 10 and 11 allows single handed control. The control handle can be attached or unattached as described above with respect to handle 52.

Note the slider 356 is supported in a different housing than slider 354 which advances the instrument 350 through channel 348 of hub 332. Thus, as shown, the knob of slider 354 slides within slot 354a of housing 352 while the slider 356 moves relative to the housing 352 (see FIG. 11).

In certain surgical interventions, a physician/user might need to dissect the target tissue 314. In FIG. 11, the system 300 is shown for such use. As shown, the scope 322 is advanced through hub 332 and inside of a body lumen 312 until it reaches a target tissue 314. Note the scope 322 and hub 332 are movable relative to each other to adjust the position the scope 322 extends from or is retracted within hub 332.

Once the scope 322 with hub 332 are in position, the slider 356 of control handle 352 is advanced moving the distal portion 336b of instrument guide (instrument receiving channel) 336 out of the confines of slot 338 of hub 332 and into an operational position. Once exposed from slot 338a, the distal portion 336b of instrument guide 336 curves inwardly toward the target tissue 314. (Note in alternate embodiments the distal portion 336b could curve/deform outwardly and then rotated to be directed inwardly). The instrument 358 is inserted though slot 364 of control handle/slider 356 until its distal end 358a is advanced distally of the distal end 336b of the instrument 336 (out the distal opening of guide 336 following the curve of guide 336). The position is adjusted such that the instrument 358 can aim at the target tissue 314 and grab it for manipulation. This, as can be appreciated, in this as well as the other embodiments, the bending of the instrument guide (either pre-bent) or by a mechanism such as links 36 or 236 effects bending of the endoscopic instrument as it passes through the bend of the guide.

Slider (actuator) 354 of the control handle 352 is advanced, either prior to after advancement of the instrument guide 336 and instrument 358, to deploy the tissue stabilizer 350 similar to stabilizer 50 such that its working end 350a is advanced out of the confines of channel 348 and port 346 of hub 332 and positioned near the target tissue 314. When the instrument 358 lifts the target tissue 314, the stabilizer 350 creates pressure/counter tension on the body lumen 312. This facilitates dissection. The position of the scope 322 can be adjusted relative to the hub 332 at this time or during other times of the procedure.

A snare instrument 361 (or other dissecting instrument) is inserted into the tool channel 324 of the scope 322 and advanced distally out the channel 324. (Note the tool channel of the endoscope is also referred to herein as the working channel). Once the target tissue 314 is stabilized with the stabilizer 350, the user places the snare 361 over the target tissue 314, then pulls the target tissue 314 into the snare 361 with the instrument 358. By pulling the snare 361 backward, the user cuts the target tissue 314. Therefore, the system 300 provides for instruments for tissue stabilization and grasping outside the working channels of the endoscope while being positioned adjacent the endoscope via the mounted hub 332.

It should be appreciated that the foregoing instruments are examples of some of the instruments that can be inserted through the channels of the hub and the endoscope. Thus, in this embodiment, as well as any of the other embodiments disclosed herein, other instruments than those illustrated can be utilized.

Spacers can be provided to keep all instrument channels and conduits organized. Note that one or more of such spacers can be used with the other systems disclosed herein. More specifically, as shown in FIGS. 10 and 11, distal spacer 341 and proximal spacer 343 are provided on system 300, however, additional spacer(s) between the distal spacer 341 and the proximal spacer 343, and/or proximal of the proximal spacer 343 and/or distal of the distal spacer 341, are also contemplated. These spacers provide additional support and improve pushability and torqueability of the instrument channel 336 and the channel 344. These spacers can also provide additional support for the channel 348. The spacers 341, 343 can be cylindrically shaped and have channels through which the instrument channels and tubular portion 336 extend as shown in FIG. 10 and also include a central channel through which the endoscope 322 extends. The spacers 341, 343 can help restrict flexing or lateral movement of the channels and tubular portion.

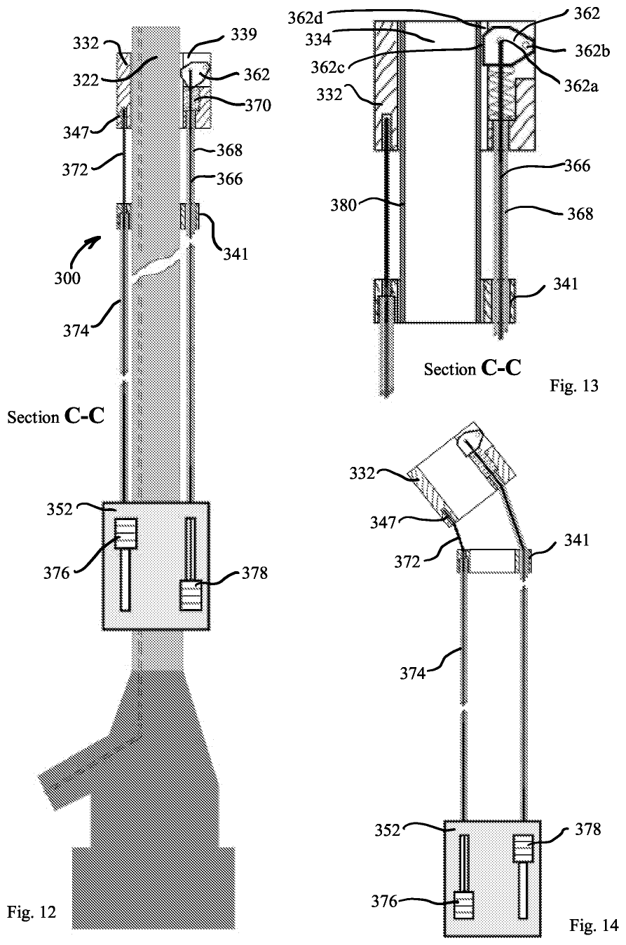

In some embodiments, a scope locking mechanism can be provided such as that shown in FIGS. 12 and 13. The scope locking mechanism secures the hub to the endoscope so they move together. As illustrated, the hub 332 has a slot 339 for a scope locking mechanism, which includes a scope compression element 362. The slot 339 can be adjacent slot 338 (see cross-sectional view of FIG. 9) although other positions are also contemplated. The compression element 362 is loaded by a spring 370 creating a compression force that locks the hub 332 to the shaft of the scope 322 as it presses against the scope 322. When locked, the scope 322 and the system 300 move together. This is especially useful during the insertion of the scope 322 into the body lumen 312 so that the scope 322 delivers the system 300 to the target tissue 314.

A pull cable 366 is located within conduit 368 and attached to the compression element 362 at the attachment point 362a. The cable 366 is operated by a slider (actuator) 378 of the control handle 352. When the cable 366 is tensioned by moving the slider 378 proximally to the position of FIG. 12, the spring 370 is compressed, and the compression element 362 rotates relative to the pivot point 362b (e.g., counterclockwise). This disengages the compression element 362 from the shaft of the scope 322 as surface 362c is no longer in contact with the external wall of scope 322, but instead, the angled surface 362d faces the wall of the scope 322, thereby allowing the scope 322 and the system 300 e.g., instrument receiving channel or guide, to move independently. When the cable 366 is not tensioned and the spring 370 is not compressed, the compression element is in the position of FIG. 13 wherein the surface 362c of compression element 362 presses against the scope 322 to engage the scope 322 so the scope 322 and system 300 are “linked” or “tied” and move together.

The conduit 368 could be a coil cable casing to provide a casing for pull cable 366. Unless it is compressed, the coil 366 is very flexible making it easy to advance it through the torturous anatomy of a body lumen. But when the tension is applied to the cable 366 to disengage the hub 332 from the scope 322, the coil 366 gets compressed making it stiffer. This improves pushability, when the hub 332 needs to be moved.

An optional tubular liner 380 can be provided within hub 332 as shown in FIG. 13. The liner 380 is preferably flexible and connects the channel (opening) 334 to spacers 341. The liner 380 provides a “hub extension” as liner 380 extends proximally of the hub portion (shown in cross-section in FIG. 13) to extend the length of the opening 334 in the hub 332 for the endoscope and allows a distal end of the scope 322 to move proximally to the hub 332 without disengaging from the channel 334.

The system 300 could optionally be equipped with a hub articulation system as shown in FIG. 14. A cable 372 is located within a conduit or channel 374 and attached to the hub 332 at the attachment point 347. The cable 372 is operated by a slider 376 of the control handle 352. When the slider 376 is moved backward, it tensions the cable 372. This action articulates the hub 332 from a linear position to an angled position with respect to the longitudinal axis as shown in FIG. 14. Such hub articulation can be provided in the other systems disclosed herein.

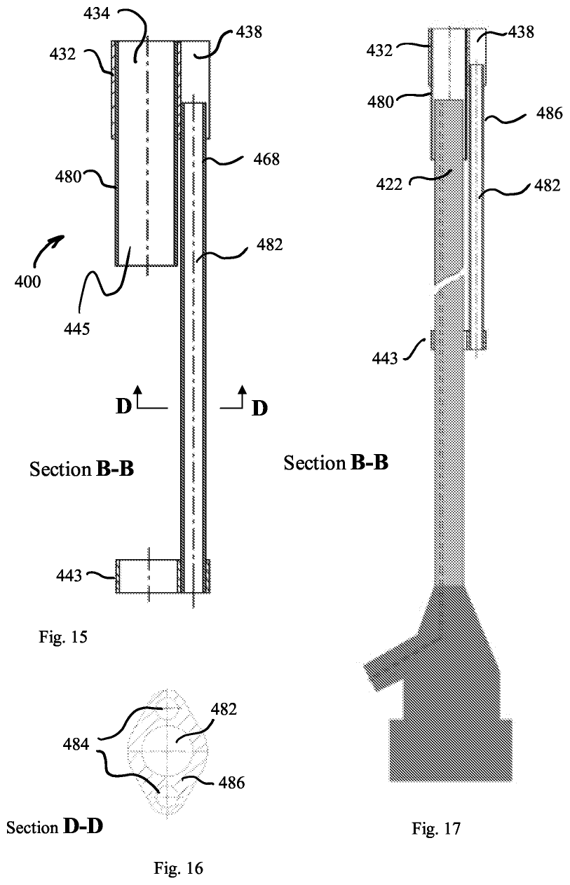

An alternative system 400 that is shown in FIG. 15-19 has a distal hub 432. The hub 432 has a cross section that is similar to the cross section of the hub 332 of the system 300 described above and shown in FIG. 9. Similarly to the system 300, which is equipped with a compression element 362, the system 400 is also equipped with at least one (but preferably two simultaneously operated) compression element(s), configured and functioning like compression element 362, for locking the system 400 to the scope 422. The compression elements are operated by pull cables in the same manner as compression element 362 is operated by pull cable 366 in the embodiment of FIG. 12. The pull cables are located with a coil cable casing 468 within lumen(s) 484. The pull cables and compression element(s) are not shown in FIGS. 15-19 as they are identical to compression element 362 and pull cable 366 for engaging and disengaging the hub and scope 422.

A flexible tubular liner 480 can be provided in some embodiments. The liner 480 is connected to the scope channel (opening) 434 and extends proximally of the hub 432 to provide a “hub extension”. This allows a distal end of the scope 422 to move proximally to the hub 432 within channel 485 of liner 480 without disengaging from the hub 432.

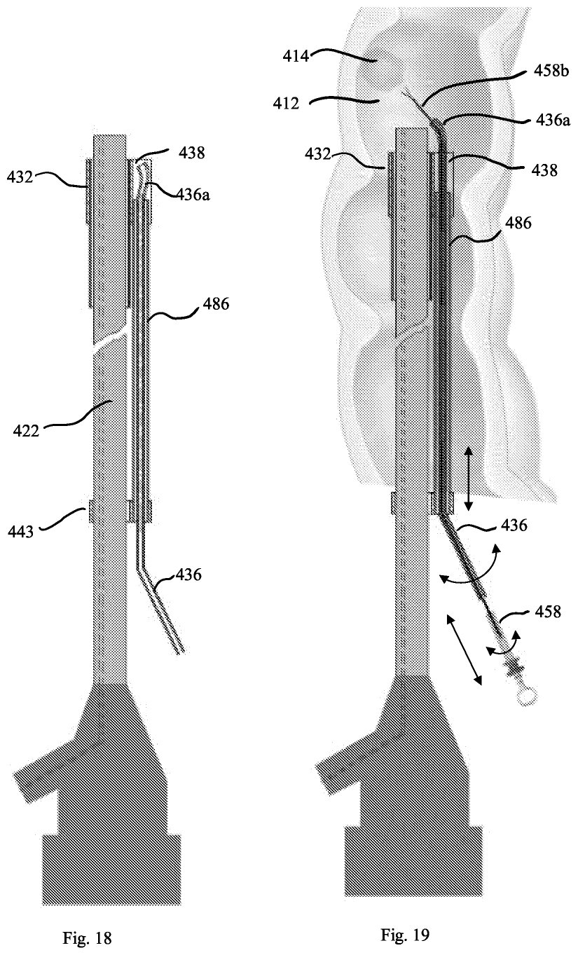

A multi-lumen conduit 486 is attached to the slot (conduit) 438 of hub 432, external of the scope 422, and extends from the hub 432 to a proximal spacer 443 (FIG. 17). FIG. 16 shows a cross-sectional view of the conduit 486. The conduit has a lumen 482 for a tool channel 436 (FIG. 18) and lumen(s) 484 for the coil cable casing(s) 468 and the cable(s) 466 for the compression elements 462. The conduit 486 is preferably made of a flexible material.

When the system 400 is placed over the scope 422, the compression elements 462 keep the system locked to the scope. Since the cables 466 are not under tension, the coil cable casing 468 remains flexible, therefore the conduit 486 remains flexible as well. This makes the insertion of the system 400 into a torturous body lumen 412 easier. When the scope 422 with the system 400 reach the target tissue 414 (FIG. 19), the cable within casing 465 is tensioned to move the compression elements 462 and disengage the system 400 from the scope 422 in the same manner as compression elements 362. Under the tension of the cable, the coil cable casing 468 is compressed and becomes stiffer. This stiffness provides additional support to the conduit 486, which makes the insertion of the instrument channel 436 easier (FIGS. 18 and 19).

Once the instrument channel/instrument guide 436 is inserted into channel 486, such that its distal tip 436a advances distally relative to the hub 432, a flexible instrument 458, such as a grasper, is inserted. By moving and rotating the instrument channel 436, the position of the distal tip 436a is adjusted such that the tip of the grasper 458b could be directed at the target tissue 414. The instrument 458 can be moved axially and rotated independently from or simultaneously with the instrument channel 436 offering users a substantial opportunity to manipulate the target tissue 414 and facilitate the resection. The arrows in FIG. 19 depict such movement.

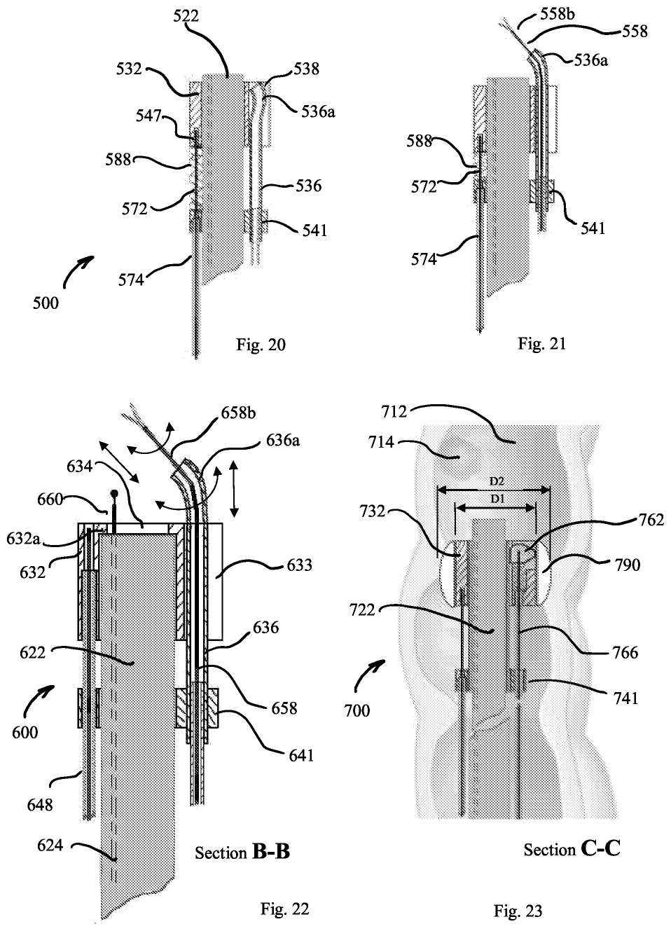

FIGS. 20 and 21 show a system 500 with an alternative mechanism for advancing an instrument channel (guide) 536. The instrument channel 536 can be the same as the instrument channel 436 or other instrument channels described herein. In this system, the spacer 541 advances the instrument channel 536.

More specifically, the system 500 includes a hub 532 with a slot or channel 538. The instrument channel 536 is attached to a distal spacer 541. The distal end 536a of the instrument channel 536 resides in the slot 538. A cable 572 is located within a conduit or channel 574 and is attached at a distal end to the hub 532 at the attachment point 547. A spring 588 maintains a predetermined distance between the hub 532 and the spacer 541. When the conduit 574 is advanced using the cable 572 for counter tension, it compresses the spring 588 (FIG. 21) and moves the spacer 541 forward. This also advances the distal end 536a of the instrument channel 536 forward from its retracted position of FIG. 20 to an extended (advanced) position of FIG. 21, angling toward the target site, for positioning the distal end 558b of a flexible endoscopic instrument 558, such as a grasper, extending therethrough, toward the target site.

FIG. 22 shows an alternative system 600 of the present invention. The system is similar to the system 300 described above, however, instead of the hub 332 with the channel 334 that allows the endoscope 322 and the hub 332 to move relative to and independently of each other, the cap 632 of the system 600 is slidably connected to the scope 622, such that both move together. The cap 632 has an aperture 634 to allow an instrument 660, such as an electrosurgical knife to move through the working channel 624 of the scope 622 and exit through the aperture 634. The cap 632 includes a wall 632a that blocks distal advancement of the scope 622. The instrument channel 636, positioned in a slot or channel 633 of cap 632 and configured to receive a flexible endoscopic instrument, can move axially and rotate relative to the cap 632. The distal end 658b of the endoscopic flexible instrument 658 can also be moved and rotated relative to the instrument channel 636 and independently of it. A spacer 641 can help retain the instrument channel 636. The cap 632 can also include instrument channel 648 for passage of an instrument such as a tissue stabilizer like stabilizer 50 discussed above.

The alternate system 700 shown in FIG. 23 is similar to the system 300 that is described above. The system 700 differs from system 300 in that it has a balloon 790 that is mounted on the hub 732. Alternatively, the balloon can be mounted on the spacer 741 or in the space between the hub 732 and the spacer 741. When inflated, the balloon 700 increases the size/diameter (transverse cross-sectional dimension) of the distal portion of the system 700 from D1 to D2. This provides for a greater stability of the system 700 relative to a body 712 and target tissue 714 by restricting movement of the system 300 and endoscope 722. Compression element 762 and cable 766 releasably lock the hub 732 to endoscope 722 in the same manner as compression element 362.

In addition, the balloon 700 could facilitate use of a technique that is called Colon Shortening. This technique facilitates fast and successful colonic intubation. Initially, the scope 722 and the system 700 are interlocked by means of a scope compression element 762 (in the same manner as compression element 362 described above) so that both could be inserted into a body lumen 712 together.

Once advancement within the colon becomes challenging, the balloon 790 is inflated creating an “anchor” point for the system 700. The scope 722 is unlocked from the system 700 by rotation/release of the compression element 762 (via pull cable 766 in a similar manner as discussed above for pull cable 366 and compression element 362) and the scope 722 is advanced forward relative to the system 700/hub 732. The balloon 790 is then deflated, and the system 700 is advanced over the scope 722 while scope 722 remains stationary. These steps could be repeated as necessary until the scope 722 and the system 700 advance to the target tissue 714.

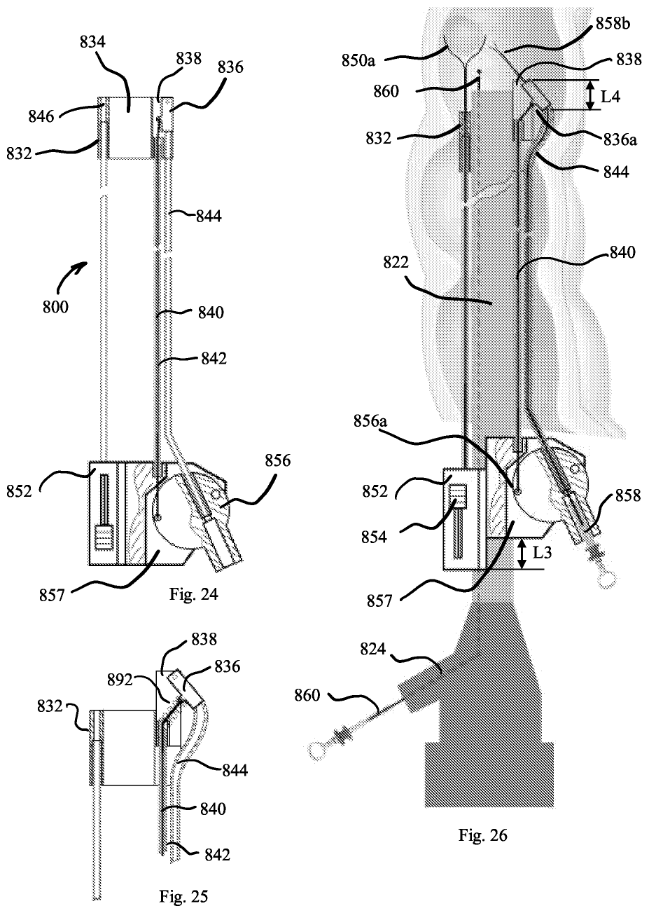

In the embodiment of FIG. 8, the link 236 extends distally of the cap 232 (or extends distally of the hub in other embodiments). In the alternate embodiment of FIGS. 24-26, the link 836 of system 800 does not extend distally. More specifically, the system 800 is similar to the system 200, however, link 836 in the retracted position and port 846 do not extend distally of the hub 832 (see FIG. 24). The link 836 is mounted on a slider 838. The slider 838 is attached to conduit 842 which is attached to axially slidable handle 857 which moves relative to control handle 852. The slider 838 is advanced by moving the sliding handle 857 distally to thereby advance the conduit 842 (see FIG. 26). Moving sliding handle 857 by a distance L3 results in the slider 838 advancing by a distance L4. In preferred embodiments L3=L4).

The link 836 is rotated by pushing on a push-pull cable 840 that is located within the conduit 842 of hub 832 and is attached to link 836 at attachment 836a and handle 856 at attachment 856b. Push-pull cable 836 is actuated by rotating the handle 856 relative to the control handle 852. Optionally, a spring 892 (FIG. 25) could also be used to facilitate this rotation. Rotation of the link 836 deforms (bends) the instrument channel 844 attached to link 836. Instrument channel 844, with its curve/bend, guides a distal end 858b of an endoscopic instrument 858 towards a target tissue 814. A stabilizer 850, like stabilizer 50 described above, is advanced by moving the slider 854 distally, such that its distal end 850a is advanced to stabilize the target tissue 814 for dissection. The dissection is performed by a cutting instrument 860, like cutting instrument 60, that is deployed via a working channel 824 of the scope 822.

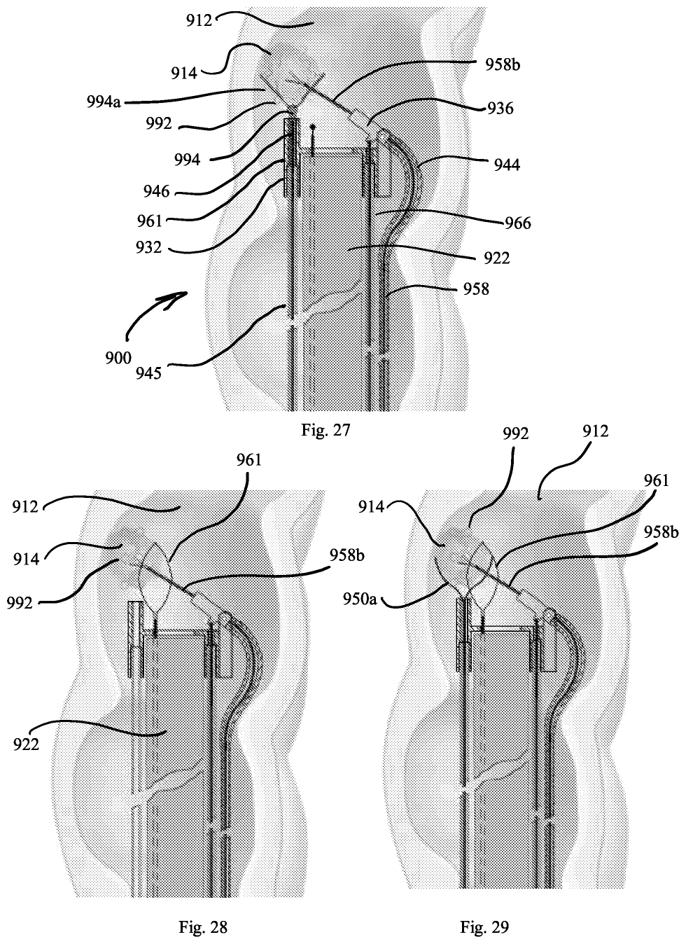

An alternative system of the present invention is shown in FIGS. 27-29. The system 900 is similar to the system 10, however, system 900, in addition to tissue dissection, could also be used for delivery of an endoscopic clip 992. The endoscopic clip that can be delivered in the illustrated embodiment is normally in the closed position supported within jaws 994a of delivery instrument 994. Examples of clips that can be delivered are disclosed in application Ser. No. 16/772,454, filed Jun. 12, 2020, the entire contents of which are incorporated herein by reference. Note the clips can be placed at an angle, for example 90 degrees, relative to the endoscope 922 such as clip 992 of FIG. 27. Thus, the clip can be angled without angling the endoscope 922. Angles other than 90 degrees are also contemplated.

The hub 932 has an instrument channel 944 and another instrument channel 945 radially spaced from each other. Channels 944 and 945, like the other hub/cap instrument channels disclosed herein, are radially spaced from the opening of the hub 932 which receives the endoscope 922 and thus the channels 944 and 945 are external of the endoscope 922, i.e., radially spaced from the external wall of the endoscope 922.

The delivery instrument 994 for clip 992 (with the jaws 994a in the closed position) is introduced via an instrument port 946 of the cap 932 of system 900 which is connected to channel 945. The instrument 994 opens a normally-closed clip 992 by separating the arms of the clip as the jaws 994a of the instrument 994 are opened. An endoscopic instrument 958 is introduced via the flexible instrument channel (guide) 944 and guided towards the target tissue 914 by articulating link 936 in a similar manner as in the system described above, i.e., via pull cable 966 pivoting link 936. A distal end 958b of endoscopic instrument 958, such as a grasper, pulls the target tissue 914 into the open clip 992. The clip 992 is then closed when released from the jaws 994a compressing the target tissue such as, for example, a perforated colon.

Another example of use of the system 900 is for a cancerous lesion that is being targeted for removal by a submucosal dissection. The clip 992 is placed between the healthy mucosal tissue and the lesion, such that when the lesion is dissected, a defect from the dissection is closed by the clip. With the clip 992 clamping tissue, the endoscopic instrument 958 is used again to position the tissue above the clip for dissection. A snare 961 (FIGS. 28 and 29), similar to the snares discussed above, is advanced through a working channel of the endoscope 922 and used to dissect the target tissue 914 with assistance of the instrument 958 that pulls this tissue into the snare 961.

As can be appreciated in these methods of use, the clip placement angle can be varied by the clip applying instrument and the tissue grasper is angled inwardly toward the target tissue due to the bending/curving inwardly of the flexible instrument channel. As with the other embodiments disclosed herein, the flexible instrument channel can be bent via actuation by an actuator (active bending since user action is required) or can be pre-set with the desired bend and bends automatically when exposed from the hub or hub channel (passive bending).

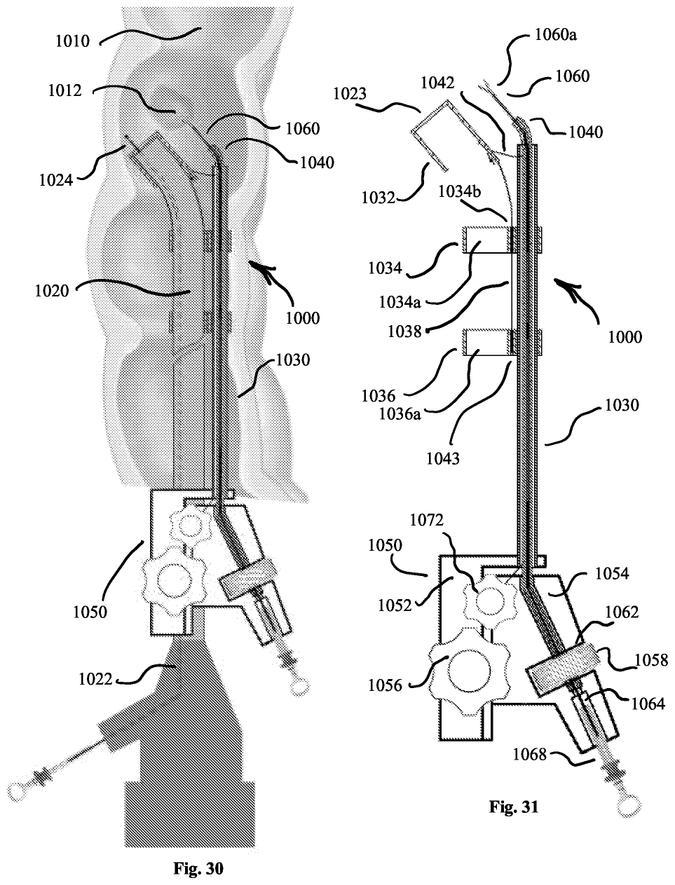

FIGS. 30 and 31 show another embodiment of a system for endoscopic resection having an external instrument receiving channel and instrument guide insertable through the channel. This embodiment provides an example of an instrument receiving channel “indirectly” receiving an endoscopic instrument via the interposed instrument guide and the instrument guide “directly” receiving the endoscope instrument thus also functioning as an instrument receiving channel. The system 1000 is mounted on a flexible endoscope 1020 and inserted into a body lumen 1010, for example a colon. The scope 1020 and the system 1000 are advanced until the distal end of the scope 1020 reaches the target tissue 1012, for example a polyp, cancerous lesion, bleeding ulcer, bleeding vessel, perforation, fistula, diverticula, etc. The scope 1020 has an instrument channel 1022 extending therethrough for insertion of a flexible endoscopic device 1024, such as a hot knife, snare or other dissecting instrument.

An external channel 1030 of system 1000 is connected to scope 1020. The external channel 1030 serves as a conduit (receiving channel) for insertion of an instrument guide 1040, which is turn serves as a delivery device for a flexible endoscopic device 1060, such as a grasper. The distal portion of the instrument guide 1040 has a curved shape, such as a C-shape or an S-shape or an L-shape. Alternatively, the distal portion could be straight or have another configuration.

The system 1000 includes cap 1032 (or hub) that is mounted on the distal end of the flexible endoscope 1020. Note in FIG. 30, the cap 1032 is shown over the endoscope; in FIG. 31, the system 1000 is shown prior to attachment of the cap 1032 to the endoscope 1022. The cap (hub) 1032 is placed over the distal end of the scope 1020 and has an aperture or opening 1023 to as not to obstruct the scope lens or obstruct instruments extending from the working channel of the scope 1022. (The caps and hubs of the other embodiments disclosed herein likewise have an opening (or multiple openings) for passage of instruments extending from the working channel of the endoscope).

The external channel 1030 is connected to scope 1020 by one or more hubs (connectors) such as a distal hub or connector 1034 and an optional more proximal additional hub or connector 1036. More than two hubs at different distances are also contemplated. Optionally, the cap 1032 and hubs 1034 and 1036 could be connected to each other with a structural element 1038, for example a wire made from nitinol, to maintain a desired set distance between the hubs. Alternatively, each hub could be attached to the body of the scope 20 with a medical tape or by other methods for a duration of the procedure. The hubs 1032, 1034 have respective channels 1034a, 1036a therein to receive the endoscope 1022.

The system 1000 has a proximal control handle 1050 that allows a user to operate the instrument guide (instrument receiving channel) 1040 and the flexible endoscopic instrument 1060 independently. The external channel 1030 is attached to a handle base 1052, which is mounted on the proximal end of the scope 1020. Alternatively, the handle base 1052 could be connected to a bracket (not shown) that is attached to an operating table (not shown). (Yet, another alternative is for the user or a user's assistant to hold it). Whether the control handle 1050 is attached to the scope 1020 or the bracket or held by the user/user's assistant, the handle base 1052 is stabilized to allow the user to operate other elements of the system with one hand.

A slider 1054 is movably attached to the handle base 1052 for moving the instrument guide 1040 within channel 1030. (In alternate embodiments, the slider 1054 can be detached from the handle base 1052 and movable relative to the handle base 1052). The slider 1054 is moved linearly (axially) relative to the handle base 1052 by rotation of a knob 1056 via an internal rack-and-pinion mechanism (not shown) or other mechanism. The rack-and-pinion mechanism controlled by the knob 1056 prevents unintended/uncontrolled (by user) movement of the slide 1054 relative to the handle base 1052, for example, when a force is accidentally applied to the distal end of the instrument guide 1040. Other designs of the moving mechanism for the slider 1054 are also contemplated to achieve movement of the guide 1040 within channel 1030 which prevent unwanted (accidental) movement of the slider 1054 which in turn prevents unwanted axial movement of the instrument guide 1040.

The instrument guide 1040 is attached to a knob 1058 that allows the user to rotate the instrument guide 1040 relative to the external channel 1030 via rotation of knob 1058. The instrument guide 1040 and the knob 1058 are assembled to slider 1054 such that the instrument guide 1040 could also be moved linearly relative to the external channel 1030. A frictional fit between the knob 1058 and a slot 1062 in the slider 1054 (see FIG. 31) assures that the knob 1058 will not turn unintentionally, for example, when a torque is accidentally applied to the distal end of the instrument guide 1040. Alternative connections that prevent uncontrolled or inadvertent rotation of the knob 1058, for example a ratchet, are also contemplated.

A flexible endoscopic instrument 1060, for example a grasper, is inserted into the instrument guide 1040 and extends past the distal end of the guide 1040 as shown. A handle 1068 of the instrument 1060 is configured to be inserted into the channel 1064 of the slider 1054 and moves linearly and rotationally relative to it. This allows the user to both advance and rotate the jaws 1060a of the grasper 1060 to position it for optimal tissue capture. The jaws 1060a of the instrument 1060 are normally closed, for example by a proximal spring mechanism, therefore the user needs to apply force to open the jaws. Once the user removes the force, the jaws 1060a will close on the tissue and hold it firmly. A frictional fit between the handle 1068 and the channel 1064 assures that the instrument 1060 cannot accidentally rotate or move linearly without the user's input. Alternative mechanism/features that prevent uncontrolled rotation or linear movement of the instrument 1060 are also contemplated.

A link 1042 in the form of a wire, suture or other elongated member, is attached at one end (its distal end) to the cap 1032. Element 1038 is attached to cap 1032 and extends through a channel 1034b in hub 1034 and is attached to hub 1036c at end 1043. The link 1042 also preferably extends through the external channel 1030 and the proximal end of the link 1042 is connected to a knob 1072 that is mounted on the handle base 1052. By rotating the knob 1072, the user can apply a proximal force to tension the link 1042. A friction between the knob 1072 and the handle base 1052 assures that the tension of the link is fully controlled by the user and cannot change accidentally.

The stability of the control base 1052 and other system's features described above that prevent accidental movement of the system's elements relative to each other allow the user to control the system with one hand. Once a desired position of the system's elements is achieved and tissue tension is established, the user can let go of the controls and use their hands for other activities. That is, the frictional engagement of the control knob 1056 ensures when the user ceases advancement of instrument guide 1040, the guide 1040 remains in its axial position and the frictional engagement of the contact knob 1058 ensures when the user ceases rotation of the instrument guide 1040, the guide 1040 remains in its rotational position. If tissue tension needs adjustment, the user can control the system with one hand. Likewise when the user ceases movement of the instrument 1060, the instrument remains in position. In some embodiments with a movable instrument channel, when the user ceases movement of the instrument channel, the channel remains in position.

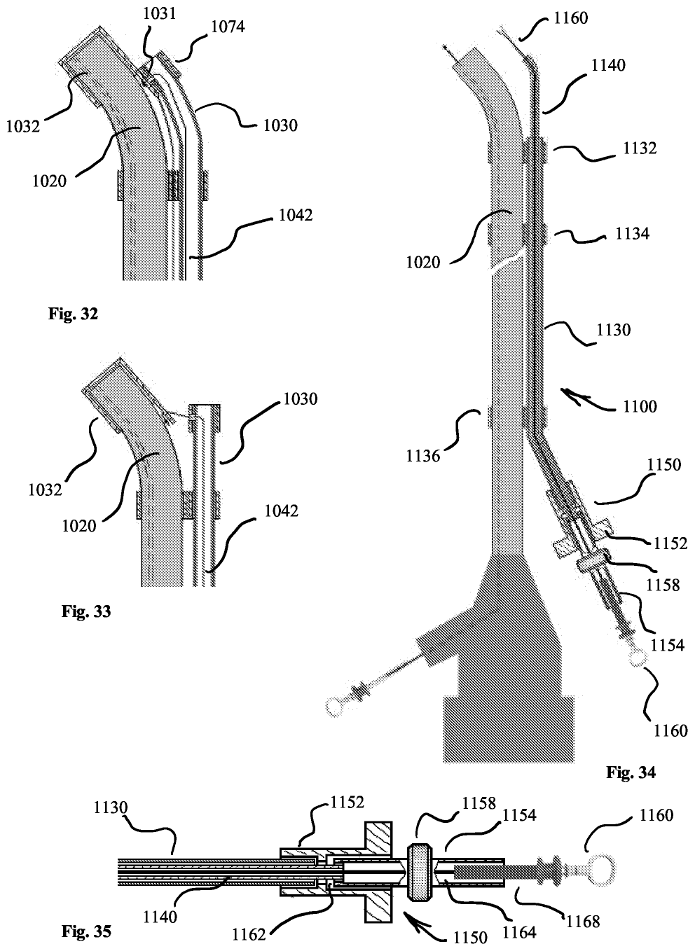

FIG. 32 shows the link 1042 extending through the external channel 1030 and exiting through opening 1031 at a distal end. An optional distal hub 1074 could be used to reinforce the distal end of the external channel 1030, and the link 1042 would also extend through the hub 1074 to attach to cap 1032.

When the link 1042 is tensioned by rotation of knob 1072, the cap 1032 and the external channel 1030 become attached to each other so when the scope 1020 is articulated, the external channel 1030 is deformed (bent/curved) and guided by the scope 1020 (see FIG. 32). In contrast, when the link 1042 is not under tension as shown in FIG. 33, the cap 1032 and the external channel 1030 are unattached and spaced further apart, and therefore the external channel 1030 doesn't get deformed (bent/curved) when the scope 1020 is articulated. Note this unattached position/condition is shown in FIG. 33 wherein the scope 1020 is articulated and the channel 1030 remains linear (unbent); the attached position is shown in FIG. 32 wherein the scope 1020 is articulated to articulate the attached channel 1030. Thus, the user can selectively decide to control the bending of channel 1030 via control of the endoscope articulation or keep the bending of channel 130 independent of the scope 1022.

FIG. 34 shows an alternative system 1100. An external channel 1130 is connected to the scope 1120 by a hub 1132. Unlike the cap 1032 of the system 1000 that is mounted on the distal end of the flexible endoscope, the hub 1132 is located proximally of the distalmost end of the scope 1120. It could be located at the portion of the scope 1120 that contains the articulating mechanism or proximal to that portion (as in FIG. 34). In this embodiment, the system 1100 relies on the scope 1120 for insertion and delivery to the target site and to provide structural support, however the scope 1120 provides no or limited assistance in guiding e.g., bending, the external channel 1130 at the target tissue. When the instrument channel 1140 is inserted into the external channel 1130, the instrument channel 1140 is supported by the scope 1120 at the hub 1132. The instrument channel 1140 has a distal tip that is configured as an L-shape, but might have another shape, for example be straight or have an S-shape, and the channel 1130 guides a flexible endoscopic instrument 1160, such as a normally-closed grasper, to the target site.

The system 1100 is operated using a control handle 1150, which allows the user to advance and rotate the instrument channel 1140 relative to the external channel 1130. It also allows the user to advance and rotate the flexible instrument 1160.

The control handle 1150 as shown in FIG. 35, has a hub 1152 that could be stabilized by being attached to a table bracket (not shown) or alternatively, it could be connected to the proximal end of the scope. Yet another alternative is for the handle 1150 to be held by the user or the assistant during the handle operation. The external channel 1130 is attached to the hub 1152. The instrument guide (channel) 1140 is attached to a sliding hub (slider) 1154. When the instrument guide 1140 is inserted in the external channel 1130, the sliding hub 1154 engages with the channel 1162 of the hub 1152. The user can move the sliding hub 1154 linearly and rotationally relative to the hub 1152 within the channel 1162 using a knob 1158 which results in the instrument guide 1140 moving linearly and rotationally relative to the external channel 1130 and to the scope 1120. The flexible instrument 1160 is inserted into the instrument guide 1140. An instrument handle 1168 of instrument 1160 is configured to move linearly and rotationally within a channel 1164 of the hub 1154. That results in a linear movement and rotation of the distal end of the instrument 1160 at the target site relative to the instrument guide 1140.

A friction fit between the hub 1152 and the slider 1154 and between the slider 1154 and the instrument handle 1168 assure that all elements of the system can move relative to each other only with the user's input. Once a desired position of the system's elements is achieved and tissue tension is established, the user can let go of the controls and use their hands for other activities as the instrument guide 1130 and instrument 1160 will remain in their axial and rotational positions.

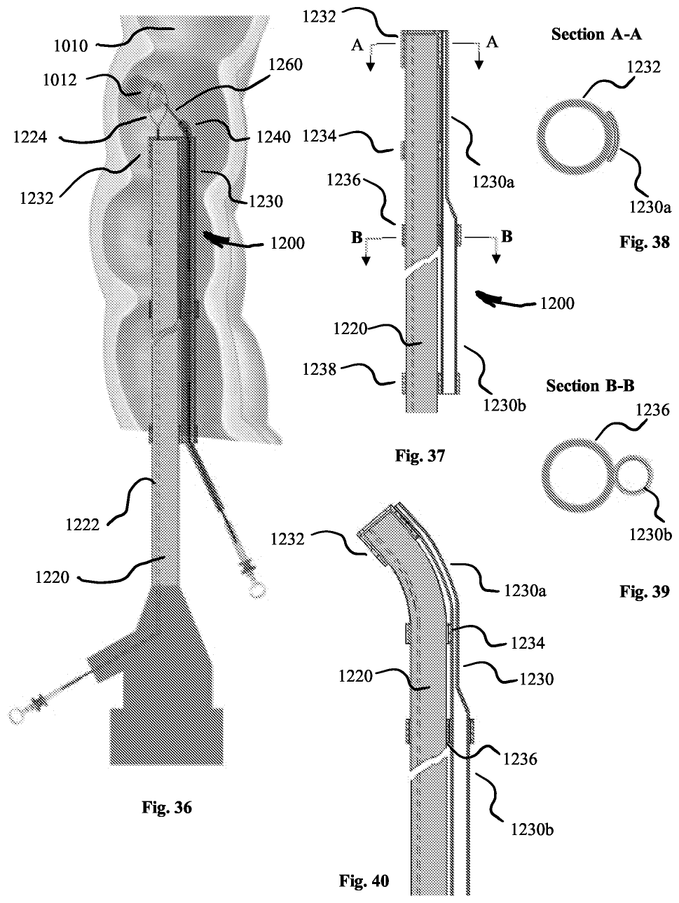

FIGS. 36-40 show an alternative system of the present invention having a collapsible channel. The system 1200 is connected to the flexible endoscope 1120 via hub or cap 1232 and is inserted with the scope 1220 into the body lumen 1010 until they reach the target tissue 1012. A flexible endoscopic device 1224, such as a snare, is inserted through the instrument receiving channel 1222 of the scope 1220 for dissecting tissue as in the snares described above. The system 1200 has an external channel 1230 and an instrument guide 1240 movable within the channel 1230. A flexible endoscopic instrument, such as endoscopic grasper 1260, is received within and movable within guide 1240. The system 1200 is connected to the scope 1220 by hubs 1232, 1234, 1236, and 1238. Fewer or larger number of hubs, as well as different locations, are also contemplated.

FIGS. 37-40 show the system 1200 before the instrument guide 1240 is inserted into the external channel 1230. Before such insertion, a distal portion 1230a of the external channel 1230 is configured to have a deformed/collapsed lumen; when the instrument guide is inserted, the distal portion 1230a expands, e.g., is stretched open. When the instrument guide 1240 is removed, the distal portion 1230a would preferably collapse again by itself to the collapsed position. The distal portion 1230a is preferably made out of a supple low-friction material. It could also have elastic/stretchable properties. In some embodiments, it could also have a reinforcing structure such as embedded/attached stent-like wire structure that would facilitate opening of the distal portion 1230a when an instrument guide 1240 is inserted.

The collapsible lumen portion can be appreciated with reference to FIGS. 38 and 39 wherein FIG. 38 shows a cross-section of the distal portion 1230a (cross-section A-A), while FIG. 29 shows a cross-section of a more proximal portion 1230b (cross section B-B). These cross-sections show the channel 1230 without the instrument guide 1240 inserted through the channel. When instrument guide 1240 is inserted through the channel 1230, the distal portion 1230a would expand, and preferably expand to a size shown in the cross-sectional configuration of FIG. 39, although a lesser or greater expansion is also contemplated. Note the size of the channel 1230 is sufficient to accept the instrument guide 1240. The remainder of the channel 1230 would preferably not change in configuration during instrument insertion, or change minimally depending on the size of the instrument. Note in alternate embodiments, insertion of the endoscopic instrument can alternatively or in addition expand the collapsible channel.

It should be appreciated that other configurations and cross-sections of the channel 1230 are also contemplated. Also, alternatively, instead of just the distal portion 1230a of channel 1230 being collapsible, additional portions of the channel 1230 could be collapsible, and in some embodiments, the entire length of the external channel 1230 could be collapsible.

The collapsed configuration of the distal section 1230a of channel 1230 reduces the profile of the external channel 1230 during the insertion of the scope 1220 and the system 1200 into the body lumen 1010. Also, such collapsed configuration makes the external channel 1230 more flexible in the direction of the main scope articulation. Therefore, when the scope 1220 is articulated, as shown in FIG. 40, the distal section 1230a, being collapsed, is more compliant, easily deformed and articulates with it.

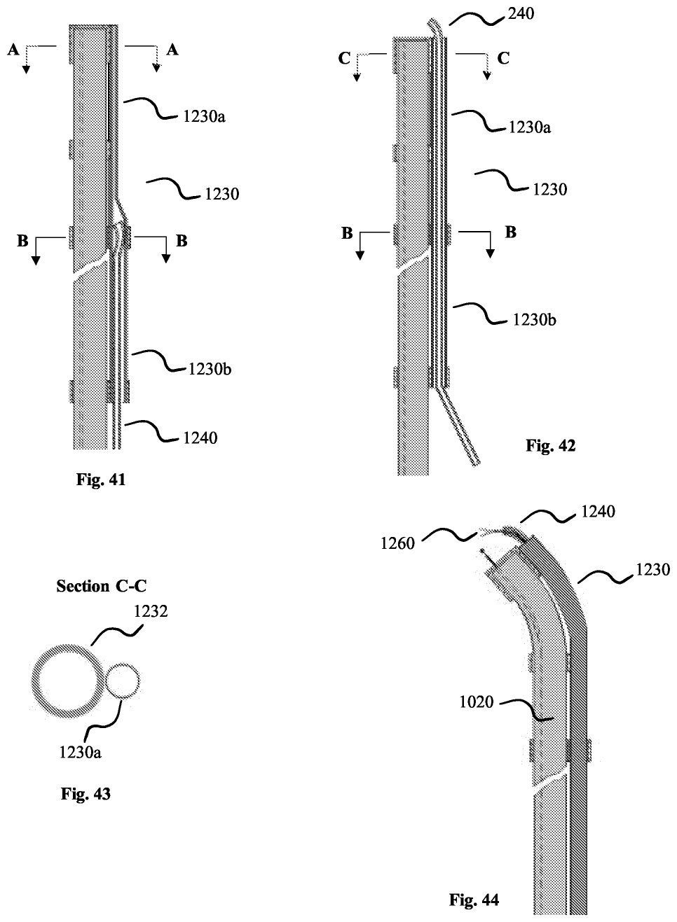

In use, with reference to FIGS. 41 and 42, instrument guide 1240 is inserted into the proximal section 1230b of the external channel 1230. When the instrument guide 1240 reaches the distal portion 1230a and continues to advance into it, the instrument guide 1240 forces the deformed/collapsed channel to expand/open up, so that the cross-section of the distal portion 130a changes from A-A (FIG. 38) to cross-section C-C (FIG. 43). When the scope 1220 is articulated, the external channel 1230, the instrument guide 1240 and the endoscopic instrument 1260 articulate/bend/deform with it as shown in FIG. 44.

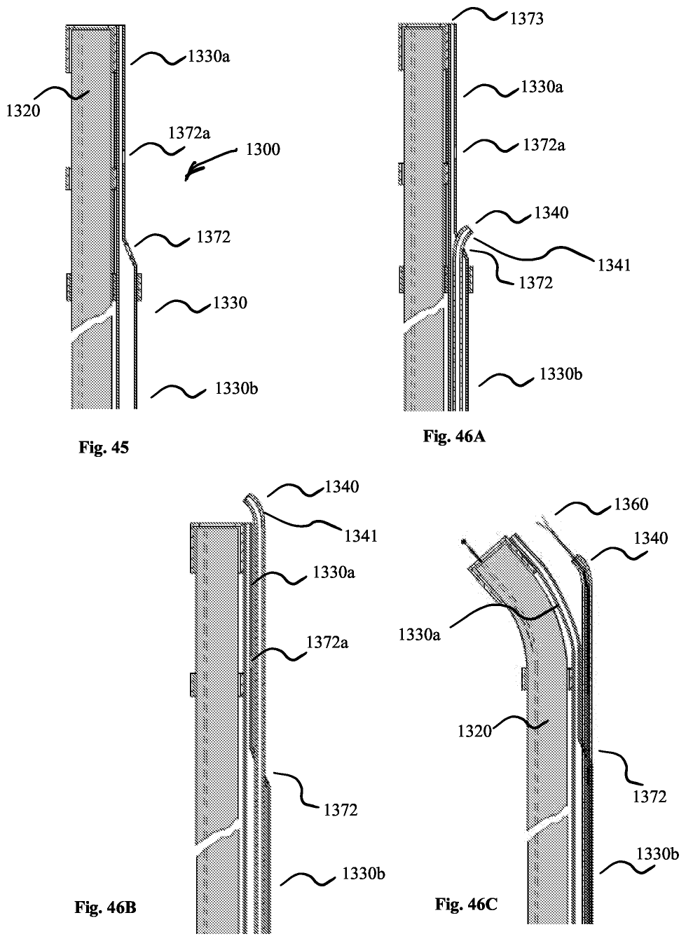

FIG. 45 shows an alternative system 1300 that is attached to the scope 1320. The system 1300 is similar to the system 1200, except that the external instrument receiving channel 1330 has a first side opening 1372. Opening 1372 is at the distal region of the channel 1330, but spaced from its distal end and spaced proximally of distal opening 1373. Opening 1372 is dimensioned to enable exit of instrument guide 1340. System 1330 can also have a second opening 1372a positioned between openings 1373 and 1372. Although two openings 1372, 1372a are provided in channel 1330 proximal of distal opening 1373, it is also contemplated that a single opening 1372 could be provided or more than two openings could be provided proximal of the distal opening 1372 for the same purpose, i.e., allowing exit of the instrument guide 1340 proximal of the distal end of the external channel 1330 at multiple locations.

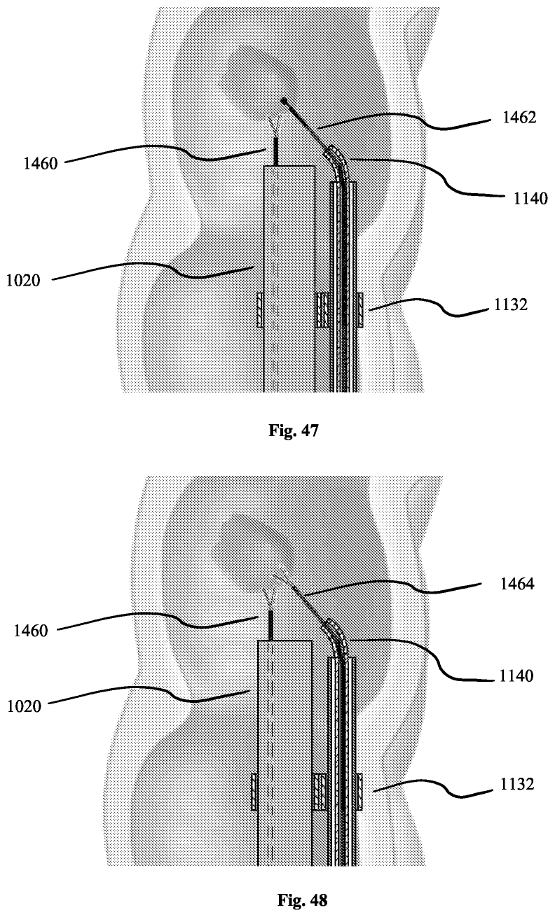

The instrument guide 1340 which forms a receiving channel to directly receive an endoscopic instrument is inserted into the proximal section 1330b of the external channel 1330. If the instrument guide 1340 is inserted with its distal tip 1341 oriented towards the scope 1220 (as in FIG. 47), the instrument guide 1340 will stay inside of the distal portion 1330a of the external channel 1330 until it exits the channel 1330 distally through the distal opening 1373 at the distalmost end of the channel 1330. However, if the instrument guide 1340 is inserted with its distal tip oriented away from the scope 1320, it will exit out of the external channel 1330 through the opening 1372 (FIG. 46). Then, when the instrument guide 1340 is advanced, it will continue out of the opening 1372 of the external channel 1330 and will be positioned outside of the distal portion 1330a of the external channel 1330. That is, it will exit the external channel 1330 before (proximal of) the distal portion 1330a. In this case, when the scope 1320 is articulated as shown in FIG. 48, the distal portion 1330a of the external channel 1330 articulates with it, however, the instrument guide 1340 and the flexible endoscopic instrument 1360 received and positioned in the instrument guide 1340 will not be affected since they are outside the articulated region of the external channel 1330. Therefore, the distal end of the scope 1320 can be manipulated/articulated independently of the instrument guide 1340 and a flexible endoscopic instrument 1360.

Thus, in the system 1300 of FIGS. 45-46C, the user can decide whether to have the instrument guide 1340 and flexible instrument 1360 articulate with the scope 1320 or to be bendable independent and not articulate with the scope 1320. This can be controlled by controlling the orientation of the distal tip of the instrument guide 1340 to selectively advance it through the side opening 1372 or the distal opening 1373. By controlling when the orientation of the instrument guide 1340 changes, the user can also choose through which one of the multiple openings 1372 the instrument guide 1340 exits the external channel 1330, thereby determining the length of the instrument guide 1340 that bends independent from the scope 1320.

When the opening 1372 is located proximally of the articulating mechanism of the scope 1320, the scope 1320 is used only for insertion and delivery of the system, but not guidance. This allows the user to articulate the scope without affecting the external channel 1330, the instrument guide 1340 or the flexible endoscopic instrument 1360. When the opening 1372 is located within the portion of the scope 1230 that contains the articulating mechanism, the scope provides some assistance with guiding of the flexible endoscopic instrument 1360 to the target tissue while still maintaining some movement independence, for example when the scope is articulated side to side. If the instrument guide 1340 is inserted all the way through the external channel 1330 without it exiting the channel via the opening 1372, the scope 1320 guides the external channel 1330, the instrument guide 1340 or the flexible endoscopic instrument 1360 to the target tissue in the same manner as shown with system 1200 of FIG. 40.

In the embodiment having an additional side opening, e.g., opening 1372a, the instrument guide 1340 is not tied to the endoscope 1230 but will partially bend with the endoscope 1230 due to its location at the articulated region of the endoscope 1230. Orientation of the tip of the instrument guide away from the endoscope will enable exit through opening 1372a if the tip is distal of opening 1372. Thus, the user can select the exit opening for the instrument guide 1340 dependent on the orientation of its tip and its axial position. Markings, e.g., lines, symbols, color coding, etc., can be provided on the proximal end of the instrument guide 1340 to indicate to the user when to rotate the tip based on axial position of the instrument guide 1340. Markings can also be provided to indicate orientation, e.g., rotational position, of the tip of the instrument guide 1340.

In the foregoing embodiments, the flexible endoscopic instrument extending through the instrument receiving channel is depicted as a grasper and the tissue dissector extending through the working channel of the endoscope is depicted as an electrosurgical knife. In the alternate system of FIG. 47, the tissue dissector 1462, e.g., snare or electrosurgical knife, for dissecting tissue as described herein is instead inserted through the instrument receiving channel 1140 and the endoscopic grasper 1460 is instead inserted through the working channel of the endoscope 1020. In the alternate system of FIG. 48, an endoscopic scissors 1464 for dissecting tissue is inserted through the instrument receiving channel 1140 and the endoscopic grasper 1460 is inserted through the working channel of the endoscope 1020. Alternatively, the endoscopic grasper 1460 can be inserted through the instrument receiving channel 1140 and the endoscopic scissors 1464 can be inserted through the working channel of the endoscope 1020. In another embodiment, both devices 1460 and 1462 are graspers. It should be appreciated that the instrument insertion of FIGS. 47 and 48 are shown by way of example in use in the embodiment of the system of FIG. 34, however, it should be understood that the depicted instrument insertion can be utilized with the other systems disclosed herein.

In the embodiment of FIGS. 47-48, the endoscope connector is in the form of a collar 1375 that is positioned around the endoscope 1320 to connect the instrument receiving channel to the endoscope. Note that this connector, unlike the hub and cap connectors described above, is positioned proximal of the distal edge of the endoscope. The connector does not form a cover like device. The connector 1375 could alternatively be positioned at regions of the endoscope other than that shown. Instead of a collar, a strap, tape or other type of connector could also be utilized to connect the instrument receiving channel to the endoscope.

Note the various structure/methods for engaging and disengaging the scope and instrument guide to tie the scope and instrument guide together or enable independent movement (articulation) of the scope and bending of the instrument guide can be utilized in each of the embodiments of the systems disclosed herein.

As can be appreciated from the discussion above, in some embodiments, the instrument receiving channel, which is dimensioned and configured to receive an endoscopic instrument has a first condition selected by a user wherein, articulation of the endoscope to an angle to a longitudinal axis of the endoscope bends a distal portion of the channel and a second condition selected by the user wherein articulation of the endoscope does not bend the distal portion of the channel such that the channel is bendable independent of the endoscope. Thus, the user can select the condition of the instrument receiving channel. In such embodiments, the instrument receiving channel can be one or more of bendable, slidable and/or rotatable with respect to the hub and/or the opening of the hub, and such movements can be independent of the positioning of the endoscope, thus providing advantages over devices which have fixed hubs and channels and systems which rely on the working channels of the endoscope or articulation/movement of the endoscope. (The opening can be considered a distal opening which allows passage of the endoscope and/or passage of an instrument extending through the working channel of the endoscope).

As can be appreciated from the discussion above, in some embodiments, the instrument receiving channel, which is dimensioned and configured to receive an endoscopic instrument or an instrument guide, can be part of the hub, can extend partially within a conduit of the hub, can be attached adjacent the hub, etc. In these various configurations, the instrument receiving channel can have the independent slidable, bendable and rotatable features described herein and such slidable and rotatable movements (various positions/conditions), and such bendable/deformable movements providing various positions/conditions (either by actuation of the user, e.g., by a linkage or pull wire, or due to a pre-bend) preferably can occur independent of the position of the scope relative to the hub.

As noted above, the movement of the instrument receiving channel, instrument guide and/or endoscopic instrument can include bending (deforming), axial movement, lateral movement, and/or rotational movement. The movement of these members could be independent of each other or alternatively dependent on one or more of another member. The movement of these members can be sequential e.g., if independent, or simultaneous, e.g., if dependent on (tied into or linked with) another member.

### Submucosal Injection Gel

Submucosal injection is a common technique to facilitate submucosal dissection. It increases visibility of target lesion margins and is designed to lower the risk of perforation. In the past, saline solution was commonly used for this purpose. However, due to time limitation, saline absorption, etc., use of alternative material for injection would be beneficial.

In some embodiments of the present invention, a pseudoplastic material that changes its flow/viscosity properties under shear stress is used as an alternative to saline solution in dissection procedures such as endoscopic resection. When the material is under pressure from the injection device, such as a syringe, it flows easily. For example, it could have a viscosity of a thin oil. When the pressure/shear force is removed, the viscosity of the material increases, for example to a viscosity of a gel, such as Vaseline.

The material is used as a delivery system for pharmaceuticals is sometimes referred to as microencapsulation. To improve visibility of the injection site, the microcapsules of the material could carry a colorant, with the ingredients of this material being safe for humans. The material can be injected via an endoscopic injection needle, such as a currently marketed Interject™ Injection Therapy Needle Catheter by Boston Scientific. The needle is inserted via a working channel of an endoscope.

Considering that the material will have oil-like viscosity during the injection via a small diameter and fairly long endoscopic injection needle, a power injection could be beneficial. Therefore, as an alternative to an injection of this material by a syringe, an injection system that is powered pneumatically, hydraulically or by a pump is also contemplated.

### Counter-Traction

When surgical tissue dissection is performed, tensioning of tissue during dissection to provide “counter-traction” to cutting is a surgical technique utilized to facilitate cutting. This technique is used in open and laparoscopic surgical procedures and would also be beneficial in the evolving field of endoluminal surgery. Examples of such endoluminal surgical procedures include endoscopic mucosal resection (EMR) and endoscopic submucosal dissection (ESD). Dissecting without proper counter-traction could limit visualization and result in adverse events, such as incomplete removal of the targeted tissue, perforations, etc. Such counter-traction is most beneficial if initiated prior to beginning the tissue dissection and continues throughout the dissection until the dissection is finished.

The present invention provides tissue tension devices for providing tissue traction to facilitate safe dissection during endoscopic surgical procedures, such as EMR and ESD. Several embodiments of the tissue tension devices are disclosed herein. In some embodiments, the tissue tension device is repositioned during the surgical procedure to maintain tension as portions of the lesion are removed. In other embodiments, the tension of the tissue tension device is adjustable without the need for repositioning. These various embodiments are discussed in detail below.

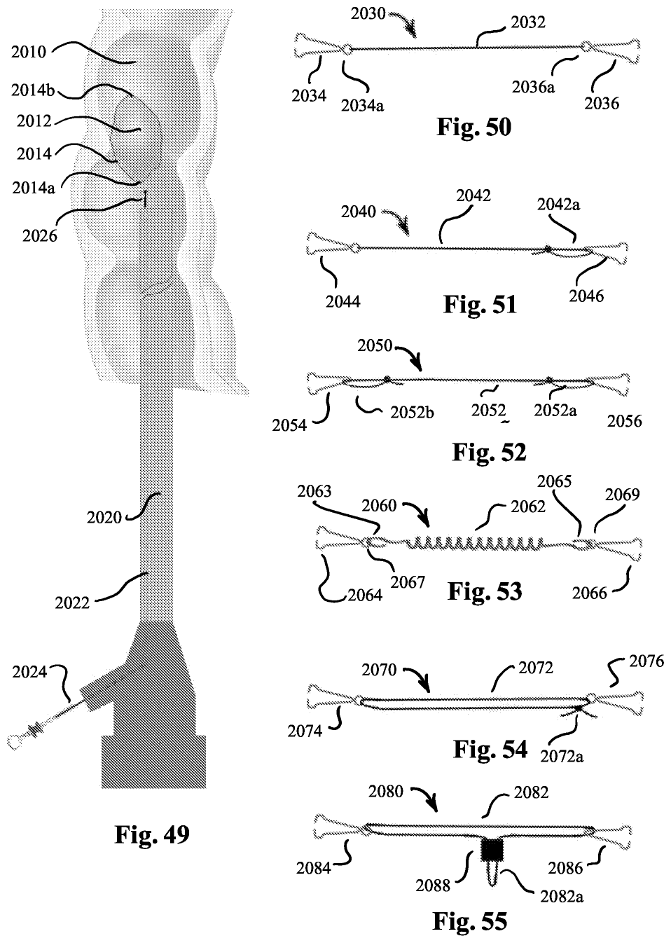

FIG. 49 shows a typical setup for procedures such as EMR or ESD. An endoscope 2020, such a flexible colonoscope, is inserted into a body lumen 2010 of a patient, such as a colon, for dissection of a lesion 2012. The endoscope 2020 has an instrument channel 2022 (also referred to as a working channel) for insertion of endoscopic instruments, such as an endoscopic knife 2024, for example Olympus America DualKnife Electrosurgical Knife. The endoscopic knife 2024 has a distal tip 2026 that interacts with the target tissue and dissects the lesion. The clinician, e.g., endoscopist, dissects the lesion 12 along an outer edge 2014 typically starting at a proximal side 2014a (near the distal tip of the scope 2020) and continuing towards a distal side 2014b (away from the distal tip of the scope 2020) by articulating and advancing the scope 2020 and manipulating the knife 2024.

Various embodiments of the tissue tension devices to aid dissection will now be discussed. With reference initially to FIG. 50, tissue tensioning device 2030 includes a tension link 2032, a lesion clip 2034 at one end of the link 2032 and an anchor clip 2036 at the opposing end of the link 2032. The clips 2034 and 2036 could have engagement or connection features such as eyelets 2034a and 2036a for attachment of the link 2032. Other methods of attaching the link to the clips are also contemplated, for example, crimp connection similar to a surgical suture-to-needle attachment.

The tension link 2032 can me made out of a metal wire. Alternatively, it could be made of a soft, non-stretchable, but strong material, such a suture or a string. It could also be made from a resilient stretchable material, such a silicone rubber. Use of a non-stretchable or a resilient stretchable material could be applicable to each of the tissue tension devices described below.

FIGS. 51-55 show alternative tissue tension devices of the present invention. FIGS. 51, 52, 53 and 54 show examples of tissue links that are non-adjustable; FIG. 55 shows an example where the tension applied by the tissue link is adjustable by the user in situ. The lesion and/or anchor clips of the links of the various embodiments in preferred embodiments can be reopened, repositioned and reclosed.

In the embodiment of FIG. 51, tissue tension device 2040 includes a tension link 2042, a lesion clip 2044 attached to one end of the link 2042 and an anchor clip 2046 attached to the opposing end of the link 2042. The clip 2044 is pre-attached to the link 2042, while the clip 2046 is not pre-attached and could be connected to the link 2042 on demand by insertion through a loop 2042a. In the alternate embodiment of FIG. 52, instead of one clip attachable on demand, link 2052 of a tissue tension device 2050 has loops 2052a and 2052b through which each of the clips 2054 and 2056 are connected on demand.

The link of the tissue tension device can be in the form of a spring as shown in FIG. 53. In this embodiment, link 2062 of tissue tension device 2060 is an extension spring. The tissue tension device 2060 has clips 2064 and 2066 that are attached by spring hooks 2063, 2065 that engage eyelets 2067, 2069 of clips 2064, 2066, respectively, at opposing ends of the spring. Other methods of attachment to the spring are also contemplated, including attachment on demand. During use, as the tissue is dissected, the tension on the spring automatically adjusts so that for certain changes of distances between the clips, sufficient tension on the tissue is maintained without needing to reposition the tension device 2060 or requiring user invention to tension or reposition the tension device 2060. Once insufficient tension occurs, the device e.g., the anchor clip, would need to be repositioned to recreate tension.

In another embodiment shown in FIG. 54, the tissue tension device 2070 has clips 2074 and 2076 at opposing ends of link 2072 that forms a loop. As an example, the loop is made of a suture that extends through eyelets of the clips 2074 and 2076 and is tied with a knot 2072a. Other suture securement is also contemplated. The link 2072 could also be made of a wire.

In the embodiment of FIG. 55, the loop is adjustable in situ. Tissue tension device 2080 has clips 2084 and 2086 attached to opposing ends of link 2082. The link 2082 could be made out of a suture or a wire and has an adjustable length. The distance between clips 2084 and 2086 could be adjusted by pulling a loop 2082a of the link 2082 through a one-way cleat 2088. This is described in more detail below in conjunction with FIGS. 61 and 62. The cleat or ferrule 2088 allows the suture or wire to be pulled in a tensioning direction, but will not allow backing out to release the tension. This can be accomplished due to a narrowing passageway, oriented teeth, etc. Other mechanisms, e.g., suture anchors, suture locks, etc. can be utilized to enable pulling in only one direction and prevent movement in the opposing direction.

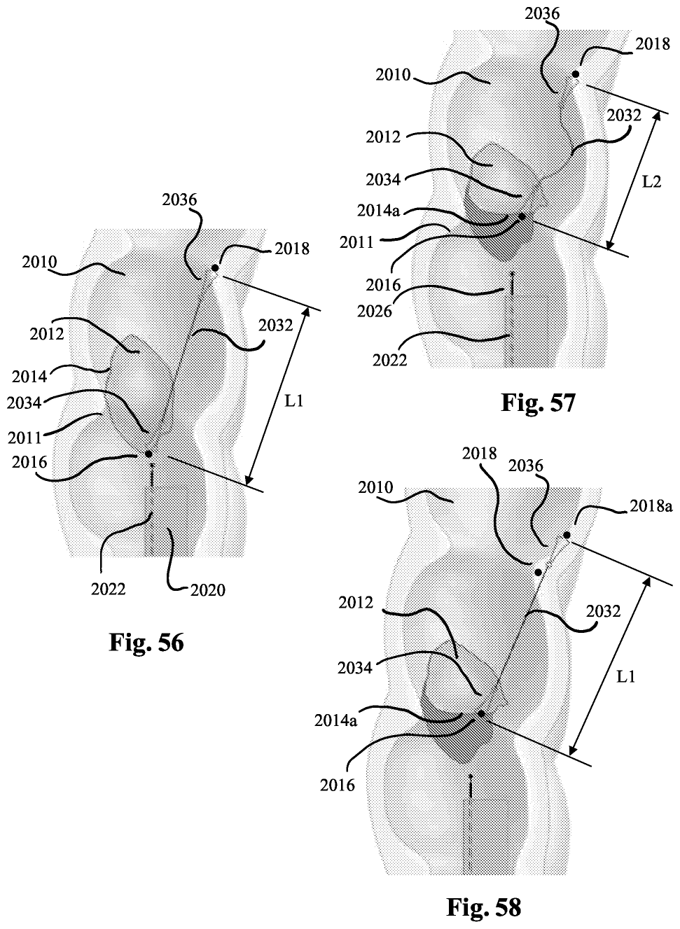

Use of the tissue tension device will now be described. Note the use of tension device 2032 will be described by way of example, it being understood that the other tissue tension devices disclosed herein can be used in the same manner. Prior to dissecting the lesion 2012 within colon 2010 along a lesion's outer edge 2014, the lesion clip 2034 is attached to the lesion 2012 at the tension point 2016 (FIG. 56). The anchor clip 2036 is then attached to the anchor point 2018 on the colonic wall such that the link 2032 is under tension establishing the distance L1 between the clips 2034 and 2036. This provides initial tissue counter-tension on the lesion 2012 when dissection of the lesion begins.

FIG. 57 illustrates tissue dissection using the distal tip 2026 of an endoscopic knife 2024 extending through and out of the working channel 2022 of the endoscope 2020. As dissection progresses, the proximal side 2014a of the lesion 2012 separates from the colonic wall 2011 of colon 2010. This reduces the distance between the tension point 2016 and the anchor point 2018 to L2, and the tension of the link 2032 is diminished or completely gone. When a non-stretchable material is used for the link 2032, the clip 2036 is repositioned from the anchor point 2018 to a new anchor point 2018a to maintain/reestablish the length L1 between the tension point 2016 and the anchor point 2018, such that tissue tension remains sufficient (FIG. 58). As tissue dissection continues, the clip 2036 can continue to be repositioned to maintain tension until dissection is complete. Use of the link 2032 that is made of a stretchable material, such a silicone rubber, or use of the device 2060 with a spring-like link 2062 instead of the device 2030 with a non-stretching link 2032 could mitigate this reduction in distance as the link will begin contracting as the distance between the clips 2034 and 2036 is reduced.

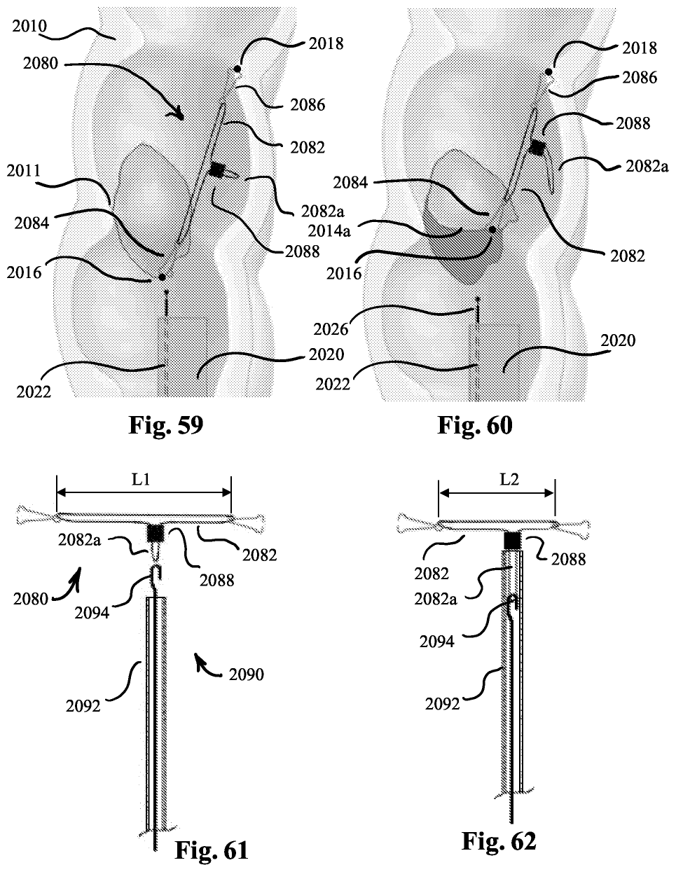

FIGS. 59 and 60 show the adjustable tissue tension device 2080 of FIG. 55 in use. Tension device 2080, as explained above, includes a tension link 2082, a lesion clip 2084 and an anchor clip 2086. The clip 2084 is attached to the lesion 2012 at the tension point 2016 and the anchor clip 2086 is attached to the anchor point 2018 on the colon wall 2010 such that the link 2082 is under tension. As dissection is performed using the distal tip 2026 of an endoscopic knife 2024 (FIG. 60) which extends through and out the working channel 2022 of scope 2020, the proximal side 2014a of the lesion 2012 separates from the colonic wall 2011. To keep the link 2082 under tension, its length is adjusted/reduced by pulling the loop 2082a though the one-way cleat 2088.

A tool 2090 (FIG. 61) provides one example of a tool that could be used for pulling the loop 2082a to tension the link 2082 of the device 2080. Other tensioning tools could also be utilized. The tool 2090 includes a link engagement device such as a hook 2094 located within the lumen 2092 of the device during delivery. The hook 2094 is movable axially within the lumen 2092 from a retracted shielded position to an extended exposed position.

In use, the hook 2094 is advanced distally out of the lumen 2092 and connected to the loop 2082a of tension device 2080. After the hook 2094 is connected to the loop 2082a, it is retracted as shown in FIG. 62 to pull the loop 2082a though the one-way cleat 2088, thereby reducing the length of the link 2082 from L1 to L2 to maintain tissue tension. In this manner, as the lesion is dissected, the clip 2086 does not need to be repositioned but can remain at its initial attachment point while the tissue remains tensioned via adjustment of the loop 2082a. The endoscopic knife 2026 could be temporarily removed if desired while the tool 2090 is inserted via the instrument channel 2022 of scope 2020 to adjust the length of the link 2082 and reestablish tension. After the tension is reestablished, the tool 2090 can be removed and the knife 2024 can be extended and dissection with the distal end 2026 of the knife 2024 can resume. Alternatively, the knife 2024 can remain in position throughout tensioning and the tool inserted through another working channel of the scope 2022.

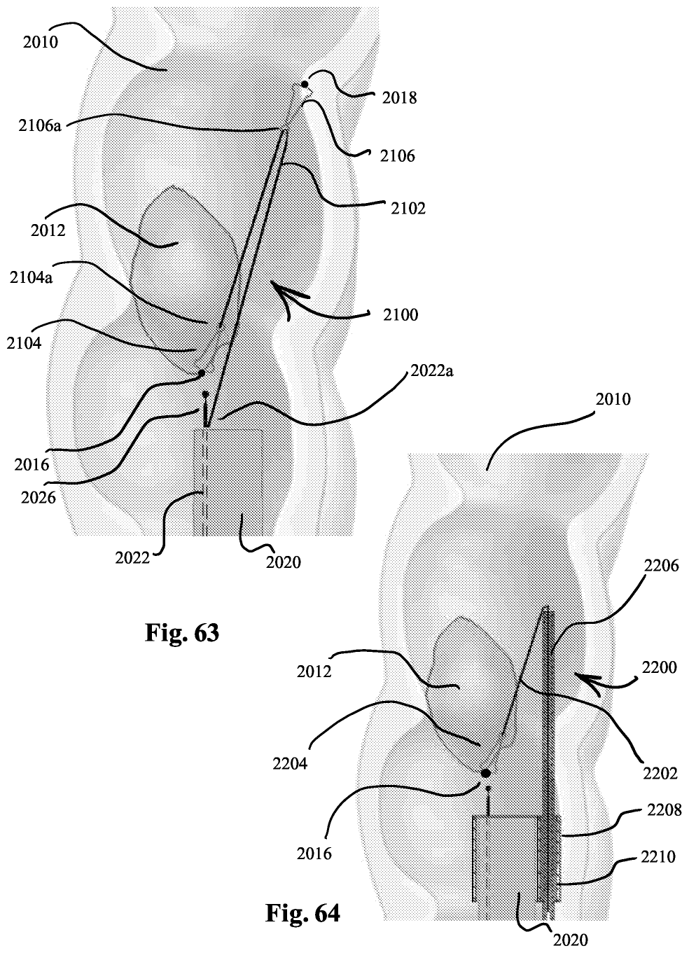

FIG. 63 shows an alternative tissue tension device 2100. The clips 2106 and 2104 are joined by link 2102. The link 2102 is fixedly connected to the clip 2104 at a connection point, e.g., eyelet 2104a. The link 2102 is connected at the other end to clip 2106 by being threaded through eyelet 2106a of clip 2106 so that it could slide relative to the clip 2106. The link 2102 (and link 2202 described below) can be in the form of a suture, wire, string, etc. and stretchable or non-stretchable material. The tensioning link goes through the working channel of the endoscope and is adjustable/tensioned from the proximal end.

Lesion clip 2104 is attached to the lesion 2012 at the tension point 2016 and the anchor clip 2106 is attached to the anchor point 2018 on the colonic wall of colon 2010. The link 2102 loops through eyelet 2016a and extends into the instrument channel 2022 of the endoscope 2020 at the channel's distal opening 2022a. The link 2102 is sized such that it could reside in the channel 2022 simultaneously with the knife 2024 and move independently from the knife 2024 and relative to the scope 2020. Alternatively, the link 2102 can extend in a separate channel of the scope 2020 so the link 2102 and knife 2024 are in different channels. The link 2102 extends to the proximal end of the scope 2020 and the instrument channel 2022, and is accessible at a proximal end where the user can pull on it to maintain tension on the lesion 2012. That is, the tensioning link 2102 extends through the working channel 2022 of the endoscope for adjusting/tensioning from the proximal end. Alternatively, the link 2102 can be attached to an actuator at the proximal end so that the actuator is moved, e.g., slid axially or rotated, to apply tension to the link 2102 and pull it proximally to tension the link 2102. In this manner, as the lesion is dissected and the tension is released, as a result of the dissection, the user can continuously adjust the tension of the link so that sufficient tension is maintained during full dissection without the need to reposition the clip 2106.

An alternate embodiment of the tissue tension device 2200 is shown in FIG. 64. In this version only one clip is provided. A hub 2208 is mounted on or near the distal end of the scope 2020. The channel or guide 2206 moves axially relative to a lumen 2210 of the hub 2208. A lesion clip 2204 is attached to the lesion 2012 at the tension point 2016. A link 2202 is connected at one end to the lesion clip 2204, e.g., via an eyelet, and the other end of link 2022 is located within the channel 2206. Link 2202 extends to the proximal end of scope 2020 wherein the user could pull on it (or activate an actuator operatively connected to the link 2202) to maintain tension on the lesion 2012. Alternatively, tension force could be controlled by moving the channel 2206 axially, i.e., the link 2202 can be attached to the channel 2206 and thereby movable with the channel 2206. Thus, in this version, like the embodiment of FIG. 63, as the lesion is dissected and the tension is released, as a result of dissection, the user can continuously adjust the tension so that sufficient tension is maintained during full dissection. Thus, in situ adjustment eliminates or at least reduces the times necessary to reposition the clip.

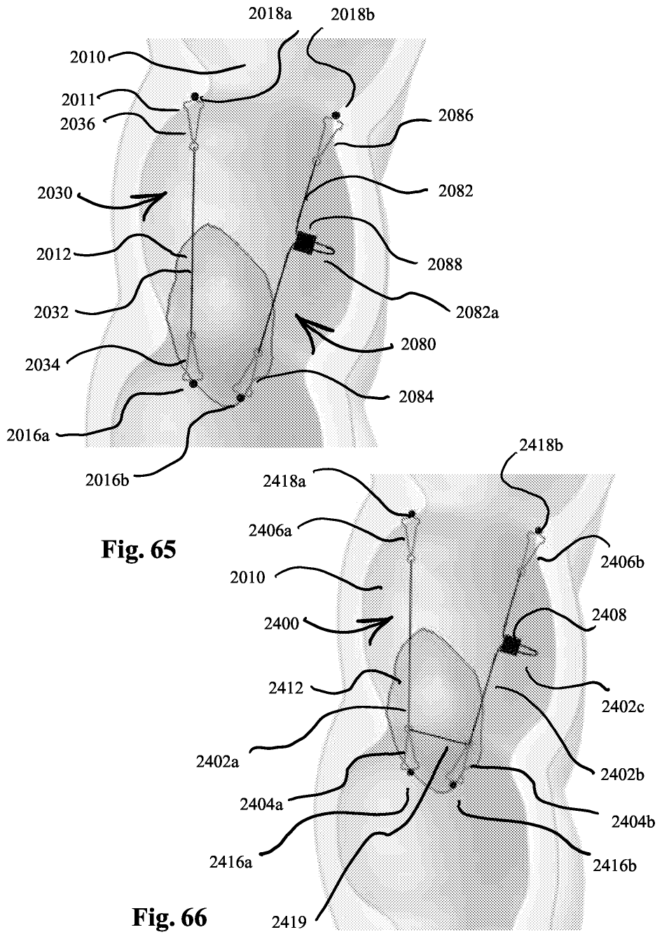

Although the foregoing embodiments disclose use of a single tissue tension device, it should be appreciated that more than one tissue tension device could be used in a surgical procedure. FIG. 65 provides an example of an embodiment utilizing two tissue tensioning devices. Tissue tension device 2030 of FIG. 50 is used in conjunction with the tissue tension device 2082 of FIG. 55. It should be appreciated that the utilization of tissue tension devices 2030 and 2082 are shown by way of example as various combinations of the tissue tension devices described herein as well as different quantities of the tissue tension devices described herein, are also contemplated.

The first tension device 2030, as described above, includes a tension link 2032, a lesion clip 2034 and an anchor clip 2036. The lesion clip 2034 is attached to the lesion 2012 at the tension point 2016a and the anchor clip 2036 is attached to the anchor point 2018a on the colonic wall 2011 of colon 2010 such that the link 2032 is under tension. The second tension device 2080 includes a tension link 2082, a lesion clip 2084 and an anchor clip 2086. The lesion clip 2084 is attached to the lesion 2012 at the tension point 2016b and the anchor clip 2036 is attached to the anchor point 2018b on the colonic wall 2011 such that the link 2082 is under tension. Tension of the link 2082 is adjusted by pulling a loop 2082a of the link 2082 through a one-way cleat 2088 using the tool 2090 as described above extending through and distally of the endoscope 2020, or alongside the scope 2020 via an instrument channel of a hub (like channel 2206 of hub 2208). Link 2082 can also be pulled by other methods. Anchor clip 2036 is repositioned as the lesion is dissected in the same manner as in FIGS. 57 and 58.

FIG. 66 shows an alternative tissue tension system. The system 2400 includes a tension link 2402 having two links or link portions 2402a, 2402b, two lesion clips 2404a and 2404b, and two anchor clips 2406a and 2406b. Additional clips and tension links are also contemplated. (The system 2400 can also be considered as having two tension links 2402a, 2402b linked by attachment link 2419).

A distal end of the link 2402a is connected to clip 2406a and a distal end of link 2402b is connected to clip 2406b. A proximal end of links 2402a, 2402b are connected to the clip 2404a and 2404b, respectively. Alternatively, this connection could be slidable with respect to the clip rather than fixed as shown. The lesion clips 2404a and 2404b are attached to the lesion 2412 at the tension points 2416a and 2416b, respectively. The anchor clips 2406a and 2406b are attached to the anchor points 2418a and 2418b, respectively, on the colonic wall of colon 2010 such that the links 2402a, 2402b are under tension. In the embodiments having a single link, extension 2419 is integral with links 2402, 2402b. In the embodiments of two separate links 2402a, 2402b, the link 2402a and 2402b are attached by a suture, wire or other connector 2419 which can be a separate suture, wire or connector or alternatively, as in the illustrated embodiment, it could be from the suture or wire which forms the links 2402a and 2402b, as it extends through the eyelets on clips 2404a, 2404b. ITension of the links 2402 could be adjusted by pulling a loop 2402c of the link 2402b through a one-way cleat 2408 (like cleat 2088 of FIG. 55) using the tool 2090 (or other instruments or methods) that was described above. Multiple cleats 2408 are also contemplated. Other mechanisms providing unidirectional movement for tensioning the link 2402 are also contemplated. Attachment (linking) of the tissue tension devices can be utilized for other embodiments of the links, e.g., the links of FIGS. 51-54.

The aforedescribed clips of the tissue tension devices are preferably normally biased in a closed position and are opened by an endoscopic instrument to surround tissue and released to return to their closed position. However, it is also contemplated that the clips could be normally in an open position and are movable to a closed position by an endoscopic instrument.

### Sensing

When a scope, such as a colonoscope, is inserted into the gastrointestinal tract, for example, a colon, a gastroenterologist no longer has any visual information about the scope shape. A colon is essentially a flexible and highly tortuous tube, and advancing the colonoscope may displace the colon and stretch the surrounding tissue, a phenomenon known as looping.

Loops, colonic elongation, and tortuosity are the most common reasons for failure to reach the cecum, which is the most distant point of the colon, even in the hands of experienced users. Ultimately, incomplete colonoscopies result in undiagnosed disease, and lead to higher incidents of colorectal cancer.

When the colonoscope is inserted into the colon, the doctors can only observe the colon anatomy in front of the camera as the scope is advanced, but they have no visual information regarding the tortuosity of the colon and could be unaware of a loop formation. The rate of incomplete colonoscopies could be as high as 25% with looping occurring in 91% of all colonoscopies. Avoiding looping by providing the doctors with a real-time information about the scope's shape and location within the colon will lead to greatly improve the colonoscopy completion rate.

Although a few colonoscopy systems offer electromagnetic shape sensors, these sensors are fully integrated and cannot be installed/retrofitted on other models of the endoscopic systems and colonoscopes. To get access to the shape sensing technology, the providers would have to acquire an entirely new system and colonoscopes. A typical gastroenterology office already owns at least one endoscopic system (cost approximately $40,000) and multiple colonoscopes (cost $43,000-$46,000 each). An incremental cost of purchasing a new system is impossible to justify and prohibitive for most gastroenterology offices and clinics.

The present invention provides a system that includes a reusable navigation monitoring unit and a simple single-use 3D fiber optic shape sensing device that could be used as needed during a colonoscopy in conjunction with any model of a colonoscope. Fiber optics shape sensing technology could be used to sense curvature and shape of a medical device in 2D and 3D. This technology could be advantageous when a user would benefit from having information about the device's shape, but cannot visualize at least a portion of this device that is inserted into a patient's body.

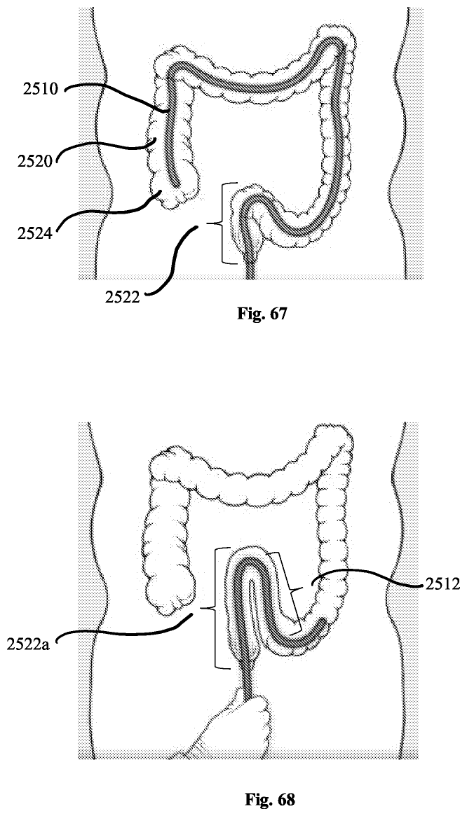

FIG. 67 shows a colonoscope 2510 inserted into a colon 2520 during a typical colonoscopy. The colon 2520 is in its natural/normal anatomical shape. The scope 2510 is inserted via the sigmoid portion 2522 of the colon all the way to cecum 2524, which is the remotest intended point of a colonoscopy, without loops. FIG. 68 shows the scope 2510 inserted via the sigmoid portion 2522a, but the sigmoid portion 2522a in this case is deformed by a colonoscope loop 12.

The shape sensing device of the present invention will provide users with a real time information about the overall shape of the inserted portion of the scope so that users become aware if the loop is forming. Knowing the location of the loop is valuable because it would allow the user to adjust the insertion technique and eliminate the loop, thereby improving chances of successfully reaching the cecum and reducing patient discomfort.

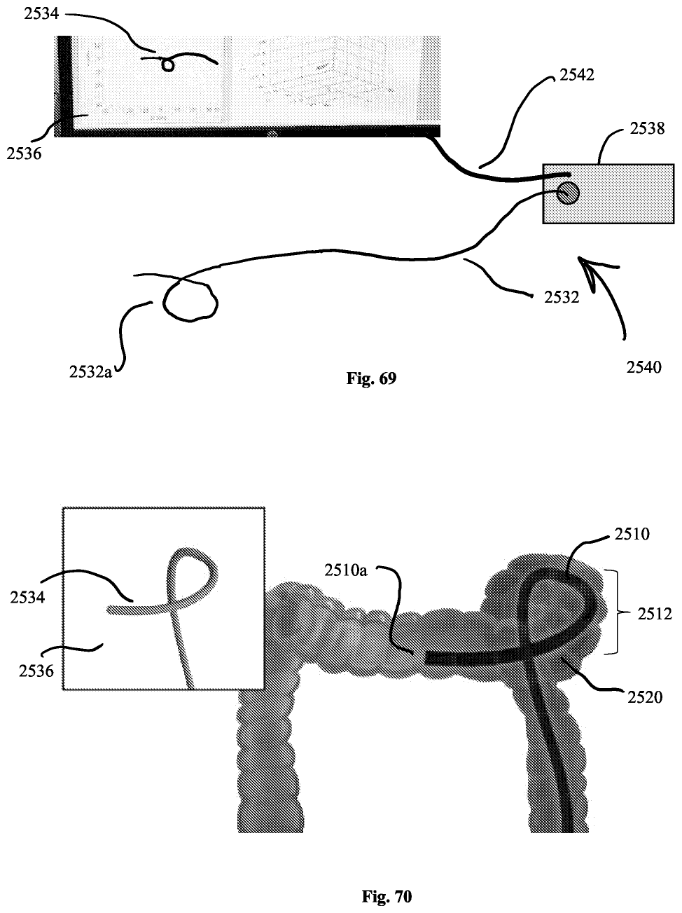

FIG. 69, shows a system 2540. A fiber optics shape sensor 2532 is deformed to form a loop 2532a. The sensor 2532 is connected to an interrogator 2538 that interprets signals from the fiber optics and displays it as an image 2534 on a digital display 2536 via a connecting cable 2542. This shows how the shape sensing technology can be effectively used to detect and provide images of the unwanted loops so corrective action can be taken.

FIG. 70 shows the endoscope 2510 inserted into the colon 2520. The sensor 2532 is integrated/built-in into the elongated portion of the scope all the way to its distal end 2510a. The user can monitor the shape of the inserted portion of the scope and track its location relative to anatomical landmarks by viewing the image 2534 on a screen 2536 and see when the loop 2512 is formed. As shown in FIG. 70, the sensor detects the loop 2512 and an image of the loop 2512 is displayed on screen 2536 to alert the clinician.

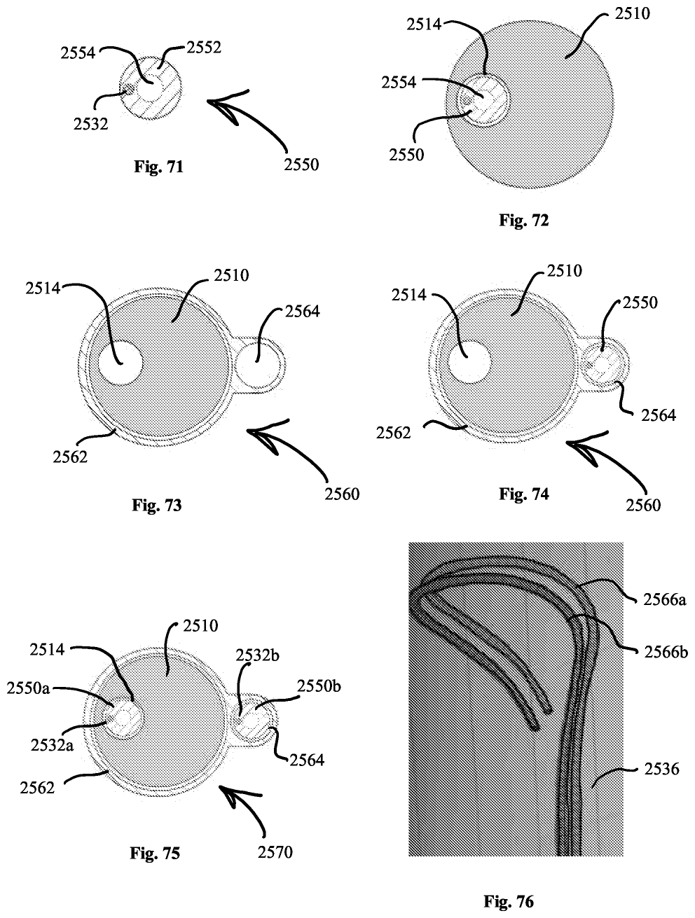

In the alternative embodiments of FIGS. 71-75, the fiber optics shape sensor 2532 is not directly integrated into the scope 2510. Instead, the sensor 2532 is part of a standalone device that could be added to the scope, when needed. Thus, in these embodiments, the loop sensing technology of the present invention can be attached to, inserted into or placed over conventional endoscopes.

FIG. 71 shows a cross-section of device 2550. The device 2550 has an elongated body 2552 with a sensor 2532 integrated within its wall. The elongated body 2552 could also have a channel 2554 for insertion of a wire mandrel, a stiffening member (described below) or a flexible instrument, such as endoscopic graspers, snares, etc. The wall thickness of the elongated body 2552 could be optimized/minimized to maximize the size (diameter) of the channel 2554, so that the channel could accommodate the largest instrument possible.

The size (outer diameter) of the elongated body 2552 could be optimized to fit into a working channel 2514 of the scope 2510 (FIG. 72) so that the device 2550 could be slidably inserted into the working channel 2514. To facilitate insertion of the device 2550, a wire mandrel (not shown) could be inserted into the channel 2554 to reinforce and improve pushability of the elongated body 2552. The wire mandrel could also be used to facilitate the insertion of the scope 2510. The wire could be inserted fully until it reaches the distal end 2510a of the scope 2510 or inserted partially. For example, once the sensing technology detects and provides images of an unwanted loop, a user inserts the wire mandrel into the channel 2554 and observes the loop uncurling when the wire crosses it. With the wire in place, the user advances the scope. After the instrument 2550 (or the scope 2510) is inserted, the wire mandrel is removed, so that the channel 2554 could be available for insertion of a flexible endoscopic instrument. Thus, in this embodiment, the sensor extends within the scope.

In an alternative system 2560 shown in FIG. 73, the system 2560 includes an elongated sheath 2562 that fits, preferably slidably, over the scope 2510. The sensor could be integrated into the wall of the sheath 2562. Alternatively, the elongated sheath 2562 could have a lumen (channel) 2564 that is dimensioned to accommodate the device 2550 to be slidably inserted into it as shown in FIG. 74. Note the channel 2564 is spaced radially from the lumen of the sheath that receives the scope 2510 and thus runs alongside and external to the scope 2510.

In some cases, it might be beneficial to use more than one sensing device 2550. The system 2570 of FIG. 75 provides an example of utilization of two sensing devices. The system 2570 has one sensing device 2550a slidably inserted into the working channel 2514 of the scope 2510. The second sensing device 2550b is slidably inserted into the lumen (channel) 2564 of the sheath 2562. This allows users to track positions of multiple elements of the system. For example, when the distal end of the sheath 2562 is aligned with a distal end of the scope 2510, and the device 2550b is fully inserted into the lumen 2564 of the sheath 2562, the position and the shape of the sensor 2532b will indicate the position of the scope 2510. The device 2550a could be partially inserted into the working channel 2514 of the scope 2510, and the position and the shape of the sensor 2532a will indicate the position of the device 2550a. As a result of using two devices 2550, the screen 2536 will display two corresponding images 2566a and 2566b (see FIG. 76).

In another embodiment, the sensing device could be integrated with the tool channel 436 of the system 400 and other embodiments described above. In this case, the device 2550a will indicate the position of the scope 422 and the device 2550b will indicate the position of the tool channel 436, thereby tracking its advancement relative to the scope 422. Integration of the device 2550b in other components of the systems described above is also contemplated.

Endoscopes, including colonoscopes, are designed for insertion into a body lumen. The body lumens, such as the colon, could have a tortuous shape. For this reason, the scopes are highly flexible, so that they could navigate this tortuous anatomy. Formation of the loops is an undesired result of that flexibility. A wire mandrel or another stiffening member could be inserted into a working channel 2514 of the scope 2510 to stiffen in for insertion. Alternatively, a wire mandrel or another stiffening member could be inserted into the lumen 2564 of the sheath 2562. Yet, another alternative is to insert a wire mandrel or another stiffening member into the channel 2554 of the device 2550.

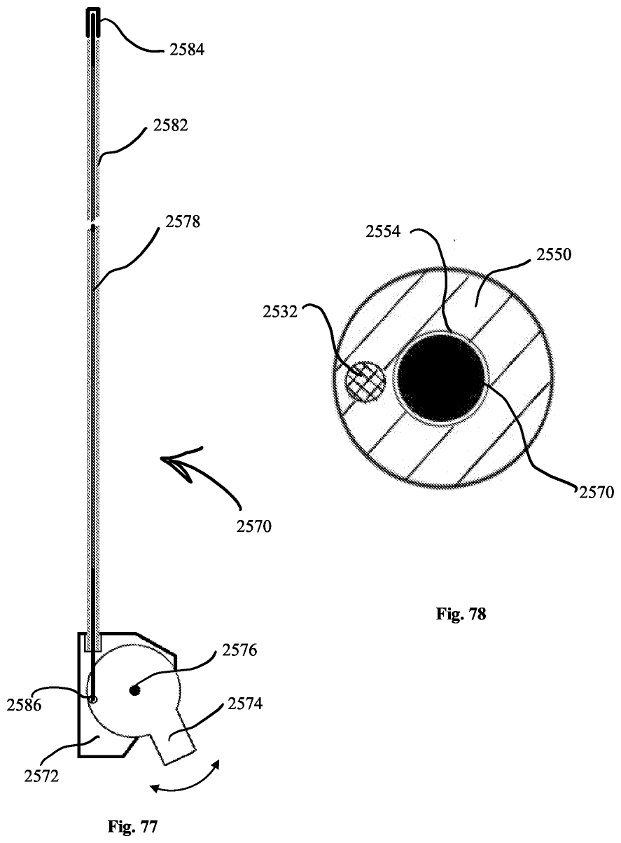

FIG. 77 shows an instrument 2570 that could serve as an alternative stiffening member for an endoscope (e.g., an alternative to the wire mandrel). The instrument has a shaft 2582 that is constructed as a wire coil. A pull cable 2578 is placed into the shaft 2582 and has a cap 2584 that is attached to its distal end. Alternatively, the pull cable 2578 could be attached to the distal end of the shaft 2582, for example by welding. The instrument 2570 also has a handle 2572 that has an eccentric lever 2574. The proximal end of the cable 2578 is attached to the lever 2574 at the attachment point 2586. Without a tension force on the cable 2578, the coil is in a relaxed state, and the instrument 2570 is highly flexible facilitating its insertion. When the lever 2574 is (e.g., counterclockwise) rotated relative to the pivot 2576, the cable 2578 is tensioned compressing the coil and making the instrument 2570 stiff.

FIG. 78 shows the instrument 2570 that is slidably inserted into the channel 2554 of the sensing device 2550. A user can prior to insertion of the scope, place the sensing device 2550 and the instrument 2570 either into the working channel of the scope or alternatively in the lumen of the sheath which has been attached to the scope prior to the scope insertion. When the user experiences a technical difficulty with insertion of a colonoscope and observes via the sensor and graphic display that a loop was formed, the user can use the device 2570 to stiffen the scope and eliminate the loop. Once the loop is eliminated, the device 2570 could be placed into the relaxed state to continue the scope insertion.

Alternatively, the user could delay insertion of the device 2550 and the instrument 2570 until the user experiences a technical difficulty. When the user experiences a technical difficulty, the user can then insert the sensing device 2550 and the instrument 2570 into the working channel of the scope or the lumen of the sheath attached to the scope until the user can locate the loop on the display. Once the sensing device 2550 and the instrument 2570 are advanced just distally to the loop, the device 2570 could be stiffened to correct the loop. The user could stiffen and relax the instrument 2570 as needed to optimize the stiffness of the scope for optimal insertion.

In some embodiments, the sensor can be incorporated directly into the endoscope. scope In one embodiment of such incorporated sensor built directly into the endoscope, the sensor is a special fiber sensor that is connected to special light source and includes receiver which receives the light as it bounces back and evaluates the light. The system can include a reader, part of or separate from the receiver, but in communication thereof, that determines what is deflected back so it knows the shape, and can communicate with a graphic display to display the detected shape of the scope.

Although the systems, devices, apparatus and methods of the subject invention have been described with respect to preferred embodiments, those skilled in the art will readily appreciate that changes and modifications may be made thereto without departing from the spirit and scope of the present invention as defined by the appended claims.

It will be understood that the above particular embodiments are shown and described by way of illustration only. The principles and the features of the present disclosure may be employed in various and numerous embodiments thereof without departing from the scope and spirit of the disclosure as claimed. The above-described embodiments illustrate the scope of the disclosure but do not restrict the scope of the disclosure and it should be understood by those skilled in the art that various changes may be made (and equivalents may be substituted) without departing from the true spirit and scope of the present invention. In addition, many modifications may be made to adopt a particular situation, material, composition of matter, process, process step or steps, to the objective spirit and scope of the present disclosure. All such modifications are intended to be within the scope of the claims appended hereto. For example, persons skilled in the art will understand that the elements and features shown or described in connection with one embodiment may be combined with those of another embodiment without departing from the scope of the present invention and will appreciate further features and advantages of the presently disclosed subject matter based on the description provided.

Where a range of values is provided, it is understood that each intervening value, to the tenth of the unit of the lower limit unless the context clearly dictates otherwise, between the upper and lower limit of that range and any other stated or intervening value in that stated range is encompassed by the present disclosure. The upper and lower limits of these smaller ranges which may independently be included in the smaller ranges is also encompassed by the present disclosure, subject to any specifically excluded limit in the stated range. Where the stated range includes one or both of the limits, ranges excluding either both of those included limits are also included in the present disclosure.

It must be noted that as used herein and in the appended claims, the singular forms “a”, “and”, and “the” include plural references unless the context clearly dictates otherwise.

Throughout the present disclosure, terms such as “approximately,” “generally,” “substantially,” and the like should be understood to allow for variations in any numerical range or concept with which they are associated. For example, it is intended that the use of terms such as “approximately”, “generally” and “substantially” should be understood to encompass variations on the order of 25% (e.g., to allow for manufacturing tolerances and/or deviations in design).

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present invention.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

Various combinations of all devices and methods described above may be utilized in the same procedure, sequentially and/or simultaneously.

1. A system for tissue resection during endoscopic surgery, the system comprising
a) an instrument receiving channel dimensioned and configured to receive an endoscopic instrument; and
b) an endoscope connector connecting the instrument receiving channel to the endoscope external of the endoscope;
c) wherein in a first condition selected by a user, articulation of the endoscope to an angle to a longitudinal axis of the endoscope effects deformation of a distal portion of the instrument receiving channel and in a second condition selected by the user, articulation of the endoscope does not deform the distal portion of the instrument receiving channel such that the instrument receiving channel is deformable independent of the endoscope.

2. The system of claim 1, wherein the first and second conditions are effected independent of an axial position of the endoscope relative to the instrument receiving channel.

3. The system of claim 1, wherein the first condition and second condition are different positions of the instrument receiving channel relative to the endoscope connector.

4. The system of claim 3, wherein the endoscope connector includes an opening and the instrument receiving channel is bendable and/or slidable with respect to the opening.

5. The system of claim 3, wherein the endoscope connector includes an opening and the instrument receiving channel is rotatable with respect to the opening.

6. (canceled)

7. The system of claim 1, further comprising an endoscope locking member selectively controllable between a locking position and a non-locking position to effect the first and second condition, wherein in the locking position the first condition is effected as articulation of the endoscope to an angle to a longitudinal axis of the endoscope deforms a distal portion of the instrument receiving channel and in the non-locking position, the second condition is effected as articulation of the endoscope does not deform the distal portion of the instrument receiving channel such that the instrument receiving channel is bendable independent of the endoscope.

8. The system of claim 1, wherein the instrument receiving channel is pre-bent and is retained in a more linear position when retracted within a conduit of the endoscope connector and moves to the pre-bent position when exposed from the conduit.

9. The system of claim 1, further comprising a link connected to the instrument receiving channel and an elongated member operatively connected to the link, wherein actuation of the elongated member effects pivoting of the link to effect bending of the instrument receiving channel.

10-16. (canceled)

17. The system of claim 1, wherein the instrument receiving channel is part of the endoscope connector and receives a flexible instrument guide, the flexible instrument guide movable within the instrument receiving channel from a retracted position to an advanced position exposed from the channel.

18-24. (canceled)

25. The system of claim 20, further comprising a clip applying instrument movable within the additional channel, the clip applying instrument comprising a pair of jaws for opening and releasing a clip carried between the pair of jaws.

26. The system of claim 1, wherein the endoscope connector comprises a hub, and the system further comprises a flexible member linking the instrument receiving channel and a portion of the hub containing an opening for the endoscope, wherein the flexible member is tensionable to interlock the instrument receiving channel and the portion of the hub containing the instrument receiving channel to effect the first condition so articulation of the endoscope bends the instrument receiving channel, and when the flexible member is not tensioned, the system is in the second condition wherein articulation of the endoscope does not bend the instrument receiving channel.

27. The system of claim 1, wherein at least a distal portion of instrument receiving channel is collapsible, wherein insertion of an endoscopic instrument or an instrument guide through the instrument receiving channel expands the instrument receiving channel and in the absence of the endoscopic instrument or instrument guide, the instrument receiving channel is in a collapsed condition.

28. (canceled)

29. The system of claim 28, further comprising at least one wire attached to the instrument receiving channel such that tensioning of the tensioning member bends the at least one wire laterally outwardly to form a tissue supporting structure.

30-31. (canceled)

32. The system of claim 1, wherein the endoscope connector comprises a hub or cap positionable over a distal end portion of the endoscope.

33. (canceled)

34. A system for tissue resection during endoscopic surgery, the system comprising an endoscope connector configured to connect to an endoscope and an instrument receiving channel, the endoscope connector connecting the instrument receiving channel to the endoscope such that the instrument receiving channel is external of the endoscope, the instrument receiving channel movable with respect to the endoscope connector and dimensioned and configured to receive an endoscopic instrument, the instrument receiving channel bendable with respect to a longitudinal axis of the endoscope such that a distal portion of the endoscopic instrument extending distally of the instrument receiving channel bends at an acute angle toward target tissue.

35-37. (canceled)

38. The system of claim 34, wherein the instrument receiving channel is bendable and/or slidable independent of the position of the endoscope within the endoscope connector.

39-44. (canceled)

45. The system of claim 34, wherein the instrument receiving channel is part of the endoscope connector and is configured and dimensioned to receive a flexible instrument guide, the flexible instrument guide movable within the instrument receiving channel from a retracted position to an advanced position exposed from the instrument receiving channel.

46-60. (canceled)

61. A system for tissue resection during endoscopic surgery, the system comprising:
a) an instrument receiving channel connectable to an endoscope;
b) an endoscope connector connecting the instrument receiving channel to the endoscope external of the endoscopes;
e) an actuator for moving the instrument receiving channel between first and second positions;
f) wherein the instrument receiving channel is retained in position when the user ceases movement of the instrument receiving channel.

62. (canceled)

63. The system of claim 61, further comprising a flexible instrument guide movable with the instrument receiving channel, the flexible instrument guide dimensioned and configured to receive an endoscopic instrument, the instrument guide bendable with respect to a longitudinal axis of the endoscope such that a distal portion of the endoscopic instrument extending distally of the instrument guide bends inwardly at an acute angle toward target tissue, wherein the instrument guide is retained in position when the user ceases advancement and rotation of the instrument guide.

64-68. (canceled)

69. The system of claim 63, further comprising a control handle for moving the instrument guide axially and rotationally, the control handle being mountable to the endoscope, wherein the endoscopic instrument is retained in an axial and rotational position when the user ceases advancement of the endoscopic instrument.

70-89. (canceled)
